SEQUENCE LISTING (11 sequences)

SEQ ID NO: 1 - DNA, 1371 bp, UNKNOWN
  Bacteria <prokaryote>   ManB (corresponding to NCBI-ProteinID ADT75633, and UniProt E0J145)
atgtcgagtttaacctgctttaaagcctatgatattcgcgggaaattaggcgaagaactgaatgaagatatcgcctggcgtattgggcgtgcctatggcgaatttctcaaaccgaaaaccattgtgttaggcggtgacgtccgcctcaccagcgaaaccttaaaactggcgctggcgagaggtttacaggatgcgggcgtcgatgtgctggatatcggcatgtccggcaccgaagagatctatttcgccacgttccatctcggcgtggatggcggcatcgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgctatcgaagcccgctttaaagccctcggcgcacctgtggaattgatcaaagtgcacaacacgccggacggcaatttccccaacggtattcctaacccgctgctgccggaatgtcgcgacgacacccgcaatgcggtcatcaaacacggcgcggatatgggcattgcctttgatggcgattttgaccgctgtttcctgtttgacgaaaaagggcagtttatcgagggctactacattgtcggcctgctggcagaagcgttcctcgaaaaaaatcccggcgcgaagatcatccacgatccacgtctctcctggaataccgttgatgtggtgaccgccgcgggcggcacaccggtgatgtcgaaaaccggacacgcctttattaaagaacgtatgcgcaaggaagacgccatctacggtggcgaaatgagcgcccaccactatttccgtgatttcgcttactgcgacagcggcatgatcccgtggctgctggtcgccgaactggtgtgtctgaaaggaaaaacgctgggcgaactggtgcgcgaccggatggcagcgtttccggcaagcggtgagatcaacagcaaactggcgcaacccgttgaggcgattaatcgcgtcgaacagcattttagccgcgaggcgctggcggtggatcgcaccgatggcatcagcatgacctttgccgactggcgctttaacctgcgctcctccaacaccgaaccggtggtgcggttgaatgtggaatcgcgcggtgatgtgccgctgatggaagaaaagacaaaacttatccttgcgttattgaacaagtaa

SEQ ID NO: 2 - DNA, 1440 bp, UNKNOWN
  Bacteria <prokaryote>  ManC (corresponding to NCBI-ProteinID ADT75634, and UniProt E8PZ98)
atgaacaataataaaattattacacctatcattatggcaggtggttcaggcagtcggttgtggcgactatcaagaattctctatccgaaacaatttcttagcctaaacggtagtcataccatgcttcaaacaacggctaatcgtctggatggtttggattgtaccaacccttatgtcatttgtaatgaacaacaccgctttatagttgctgaacagcttagaaaaatcgatagattgacttcaaagaatatcatccttgagcctgttgggcgtaacactgcccctgcaattgcattagcggcgttgctgatgtctaagtctgataaaagtgcagatgatcttatgctcgtactggctgcagatcacgttatattagatgaagaaaaattttgtaacgctgttagatcggcaattccatacgctgctgatgggaaattggtaacatttggtataattccagacaaagcagaaactggttatggttatatacatcgaggacaatatattaatcaggaagattcggatgcatttatagtgtcatcatttgttgaaaagccaaatcatgagacagccactaaatatcttgcttccggtgagtattattggaatagcggtatgtttttgtttagtgcaaatcgttatatagaggaacttaaacaatttcggcctgatattttatccgcttgtgaaaaagcaattgcttcagcgaactttgaccttgattttgtgcgtttagatgaaagttctttctctaagtgtcctgaagagtcaattgattacgctgtaatggaaaaaacaaaagacgcaattgttattccaatggatgctggctggagtgatgtcggttcatggtcttctctttgggaaattaatgataaagactcagacggcaacgtaatagttggggatattttctctcatgaaacaaagaattctttcatatatgccgaatcgggaattgttgctacagtgggagtggaaaatttagttgttgtccaaacaaaggatgccgttcttgtctcagagagaaataaagttcaggatgtaaagaaaatagtcgaacaaattaaaaattcaggtcgtagcgagcattatgttcatcgcgaagtatatcgtccttggggtaaatatgattccattgacacaggggagcgttatcaggtcaaacgtataacagtaaatcctggtgaaggactttctttacaaatgcaccatcatagggcagaacattggatcatagtttctggaactgcaaaggtgactataggttctgaaactaagattcttagcgaaaatgaatctgtttacatacctcttggtgtaatacactgcttggaaaatccagggaaaattcctcttgatttaattgaagttcgttctggatcttatttagaagaagacgatgttatccgttttcaggaccgatatggtcgcagctaa

SEQ ID NO: 3 - DNA, 1122 bp, UNKNOWN
  Bacteria <prokaryote> GMD from E. coli (GMD_EC) (corresponding to NCBI-ProteinID ADT75653, and UniProt E0J125)
atgtcaaaagtcgctctcatcaccggtgtaaccggacaagacggttcttacctggcagagtttctgctggaaaaaggttacgaggtgcatggtattaagcgtcgtgcatcgtccttcaacaccgagcgcgtggatcacatttatcaggatccgcacacctgcaacccgaaattccatctgcattatggcgacctgagtgatacctccaacctgacgcgcattttgcgtgaagtgcagccggatgaagtgtacaacctgggcgcaatgagtcacgttgcggtttcttttgagtcaccggaatataccgcagacgtcgatgcgatgggtacgctgcgcctgctggaggcgatccgcttcctcggtctggaaaagaaaacccgtttctatcaggcttccacctctgaactgtacggtctggtgcaggaaattccgcagaaagaaaccacgccgttctacccgcgatctccgtatgcggtcgccaaactgtatgcctactggatcaccgttaactaccgcgaatcctacggcatgtacgcctgtaacggtattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcaacaggaacagccggaagatttcgttatcgcgaccggcgttcagtactccgtgcgtcagttcgtggaaatggcagcggcacagctgggcatcaaactgcgctttgaaggcacgggtgttgaagagaagggcattgtggtttccgtcaccgggcatgacgcgccgggcgttaaaccgggtgatgtgattatcgccgttgatccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataa

SEQ ID NO: 4 - DNA, 966 bp, UNKNOWN
  Bacteria <prokaryote>  WcaG from E. coli (WcaG_EC) (corresponding to NCBI-ProteinI  ADT75652, and UniProt E0J126)
atgagtaaacaacgcatttttatcgctggccatcgtgggatggtcggttccgccatcacgcggcagctcgaacagcgcggtgatgtggaactggtattacgcacccgcgacgagctgaacctgctggacagccgcgcggtgcatgatttctttgccagcgaacgcattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttcttggatcgtcctgtatctacccgaaactggcaaaacagccgatggcagaaagcgagttattgcagggcacgctggagccgaccaacgagccttacgccattgccaaaatcgccgggatcaaactgtgcgaatcatacaaccgccagtacggacgcgattaccgctcagtcatgccgaccaacctgtatggcccgcatgacaacttccacccgagtaattcgcatgtgatccctgcattgctgcgtcgcttccacgaggcgacggcacagaatgcaccggacgtggtggtatggggcagcggtacaccgatgcgtgaattcctgcacgtcgatgatatggcggcggcgagcattcatgtcatggagctggcgcacgaagtctggctggagaacacccagccgatgctgtcgcacattaacgtcggcacgggcgttgactgcactatccgcgagctggcgcaaaccatcgccaaagtggtgggttacaaaggtcgggtggtttttgatgccagcaaaccggatggtacgccgcgcaaactgcttgatgtgacgcgcctgcatcagcttggctggtatcacgaaatctcactggaagcggggcttgccagcacttaccagtggttccttgagaatcaagaccgctttcgggggtaa

SEQ ID NO: 5 - DNA, 1083 bp, UNKNOWN
  Bacteria <prokaryote>  Gmd from Bacteroides fragilis (Gmd_BF)
atgattgacagaatggataaaaacgcaaaaatttatgtagccggacaccacggactggtgggttcggctatatggaaaaatctgcaggaaaaggggtatacgaatctggtgggacgcacacataaggaactggacttattggacggtgcgaccgtaaagcagttttttgatgaggaaatgccggagtacgtgtttttggctgccgcttttgtcggaggaatcatggccaatagtatctaccgtgcggactttatctataagaacttacagatacagcagaacgtgatcggagaaagtttccggcatcaggtgaaaaaactgctctttctgggcagtacctgcatctatccgagggatgccgaacagccgatgaaagaggacgtcttgctcacttctccactggaatataccaatgaaccttatgccatagccaaaatagccggactgaaaatgtgcgaaagcttcaacctgcaatatggaacgaactacatcgccgtgatgccgaccaacctgtatgggccgaacgataactttgacttggaacgcagtcatgtgctgcctgccatgatccgtaaagttcacttggcacactgcctgaaaaaaggagattgggaggccgtgcgtaaagatatgaacctgcgtccggtagaaggcatcagtggtgccaactccaacgaagagatcctccggattctccggaaatacggcattactgaaacggaggtgacactttggggaacaggaacgcctctaagggaatttctttggagtgaagaaatggcagatgccagtgtcttcgtaatggaacatgtggacttcaaggatacctacaaaaccggtgcaaaagacatccgcaactgccacatcaatataggtaccggcaaagaaattacaatccgcgaactggccggactgattgtaaatacagtcggctatcagggtgaactgacttttgacagcagtaaaccggacggaaccatgcgaaaacttaccgatccgtcgaaattgcacaacctcggatggcatcataagatcgatattgaagagggggtacagaaaatgtacgagtggtatctgggataa

SEQ ID NO: 6 - DNA, 1083 bp, UNKNOWN; identical to SEQ ID NO: 5
  Bacteria <prokaryote> WcaG from Bacteroides fragilis (WcaG_BF)

SEQ ID NO: 7 - DNA, 900 bp, UNKNOWN
  Bacteria <prokaryote> FucT from H. pylori (HpFucT)
atggcttttaaggtggtgcaaatttgcggggggcttgggaatcaaatgtttcaatacgctttcgctaaaagtttgcaaaaacactctaatacgcctgtgctgttagatattacttcttttgattggagtaatagaaaaatgcaattagaacttttccctattgatttgccctatgcgagtgaaaaagaaatcgctatagctaaaatgcaacacctccccaagctagtaagaaatgtgctcaaatgcatggggtttgatagggtgagtcaagaaatcgtttttgaatacgagcctaaattgttaaagacaagccgcttgacttatttttatggctattttcaagatccacgatattttgatgctatatcccctttaatcaagcaaaccttcaccctaccccccccccccgaaaatggaaataataaaaaaaaagaggaagaataccaccgcaagcttgctttgattttagccgctaaaaacagcgtgtttgtgcatataagaagaggggattatgtggggattggctgtcagcttggtattgattatcaaaaaaaggcgcttgagtatatggcaaagcgcgtgccaaacatggagctttttgtgttttgcgaagacttagaattcacgcaaaatcttgatcttggctacccttttatggacatgaccactagggataaagaagaagaagcgtattgggatatgctgctcatgcaatcttgcaagcatggcattatcgctaacagcacttatagctggtgggcggcctatttgataaacaatccagaaaaaatcattattggccccaaacactggctttttgggcatgagaatatcctttgtaaggaatgggtgaaaatagaatcccattttgaggtgaaatcccaaaagtataacgcttaa

SEQ ID NO: 8 - DNA, 873 bp, UNKNOWN
  Bacteria <prokaryote> Codon optimized FucT from H. mustelae (codon optimized HmFucT)
atggcacaggtcgtggacttcaagatcgtccaggtccacggcggcctcggcaaccagatgttccagtacgcattcgcaaagagcctccagacccacctcaacatcccggtcctcctcgacaccacctggttcgactacggcaaccgtgagctcggcctccacctcttcccgatcgacctccagtgcgcaagcgcacagcagatcgcagcagcacacatgcagaacctcccgcgtctcgtccgtggcgcactccgtcgtatgggcctcggccgtgtcagcaaggagatcgtcttcgagtacatgccggagctcttcgagccgagccgtatcgcatacttccacggctacttccaggacccgcgttacttcgaggacatcagcccgctcatcaagcagaccttcaccctcccgcacccgaccgagcacgcagagcagtacagccgtaagctcagccagatcctcgcagcaaagaacagcgtcttcgtccacatccgtcgtggcgactacatgcgtctcggctggcagctcgacatcagctaccagctccgtgcaatcgcatacatggcaaagcgtgtccagaacctcgagctcttcctcttctgcgaggacctcgagttcgtccagaacctcgacctcggctacccgttcgtcgacatgaccacccgtgacggcgcagcacactgggacatgatgctcatgcagagctgcaagcacggcatcatcaccaacagcacctacagctggtgggcagcatacctcatcaagaacccggagaagatcatcatcggcccgagccactggatctacggcaacgagaacatcctctgcaaggactgggtcaagatcgagagccagttcgagaccaagagctaa

SEQ ID NO: 9 - DNA, 1254 bp, UNKNOWN
  Bacteria <prokaryote> LacY from E. coli (LacY_EC)
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggttctggctgtgcactcatcctcgccgttttactctttttcgccaaaacggatgcgccctcttccgccacggttgccaatgcggtaggtgccaaccattcggcatttagccttaagctggcgctggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttctctactgcgtcgtcaggtgaatgaagtcgcttaa

SEQ ID NO: 10 - DNA, 1884 bp, UNKNOWN
  Bacteria <prokaryote> LacY from Lactobacillus delbrueckii (LacY_Ldb)
atgaagaaaaagcttgtctcacgcttgtcgtacgcggccggtgcctttggcaacgacgtcttctacgcgactctgtcaacctactttatcgtcttcgtcaccacccacctctttaatgccggtgaccacaagatgatctttatcatcaccaacttgatcaccgccatccggatcggggaagtcctgctcgaccccttgatcggtaacgccatcgaccggaccgaaagccggtgggggaagttcaagccctgggttgtgggcggggggatcatcagctcattagccctcttagccctctttaccgactttggcggcattaaccaaagcaaacccgttgtttacttagtaatcttcggtattgtttacttgattatggatatcttctactcatttaaagacactggcttctgggccatgatcccggccttgtccctggattcccgggaaagagagaagacctccaccttcgccagagtcggctccaccatcggggccaacctggtcggggtagtcatcaccccaatcatcctcttcttctcggctagcaaggccaaccccaacggggataagcagggctggttcttctttgccttgatcgtggccattgtcggcatcttgacctcaattaccgttggtcttggtactcacgaagtaaaatccgccctgcgggaaagcaatgaaaagaccactttgaagcaggtctttaaggtcctggggcaaaacgaccagctcctctggctggcctttgcctactggttttacggcctgggtatcaacaccctgaacgctctgcaactttactacttctcatacatcttaggcgatgcccgcggctacagcctgctttacaccatcaacacctttgtcggtttaatctctgcatccttcttcccatcactggccaagaagttcaacagaaatcgcctcttctacgcctgcatcgcggtgatgctgttagggatcggggtcttctccgtggccagcggttctctggccctgtcccttgttggggcagaattcttctttattccgcagcctctggccttcctggtcgttttgatgatcatctctgacgctgttgaatacggccagctgaaaactggccacagagacgaagctttgaccctgtctgtccggccattggtcgataagctgggcggggccttgtccaactggtttgtttccttgattgccttaactgccggcatgaccactggggcgactgcctcaacaattacagctcatggccagatggtcttcaagttagctatgtttgccttaccggcagtcatgctcttgatcgctgtttctattttcgccaaaaaggtcttcttgactgaagaaaagcacgcggaaatcgtcgaccagctggaaactcaattcggccaaagccatgcccaaaagccggcgcaagctgaaagcttcactttggccagcccagtctccggacaattaatgaacctggacatggttgacgacccggtctttgccgacaaaaagttaggcgacggctttgccctggtgccagcagacggtaaggtctacgcgccatttgccggtactgtccgccagctggccaagacccggcactcgatcgtcctggaaaatgaacatggggtcttggtcttgattcaccttggcctgggcacggccaaattaaacgggactggctttgtcagctatgttgaagagggcagccaggtagaagccggccagcagatcctggaattctgggacccggcgatcaagcaggccaagctggacgacacggtaatcgtgaccgtcatcaacagcgaaactttcgcaaatagccagatgctcttgccgatcggccacagcgtccaagccctggatgatgtattcaagttagaagggaagaattag

SEQ ID NO: 11 - DNA, 1092 bp, UNKNOWN
  Bacteria <prokaryote> LacI from Escherichia Coli
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacacgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagcccgccagacgcagacgcgccgagacagaacttaatgggcccgctaacagcgcgatttgctggtgacccaatgcgaccagatgctccacgcccagtcgcgtaccgtcttcatgggagaaaataatactgttgatgggagtctggtcagagacatcaagaaataaagcaggaacattagcgcaggctgcttccacagcaatggcatcctggtcatccagcggatagttaatgatcagcccactgacgcgttgcgcgagaaggttgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtatagcgttactggtttcacattcaccac

(19) United States
(12) Patent Application Publication
(10) Pub. No.: US 2025/0283130 A1
(43) Pub. Date: Sep. 11, 2025

(54) GLYCOSYLTRANSFERASE DEFICIENT CORYNEBACTERIUM FOR THE PRODUCTION OF FUCOSYLLACTOSE

(71) Applicant: GALAB LABORATORIES GMBH, Hamburg (DE)

(72) Inventors: Uma Maheswa REDDY KATHA, Hamburg (DE); Jürgen KUBALLA, Hamburg (DE)

(73) Assignee: GALAB LABORATORIES GMBH, Hamburg (DE)

(21) Appl. No.: 18/546,091

(22) PCT Filed: Feb. 11, 2022
(86) PCT No.: PCT/EP2022/053368

§ 371 Date: Aug. 10, 2023

(30) Foreign Application Priority Data
Feb. 11, 2021 (EP) 21156553.6

(51) Int. Cl. C12P 19/00; C12N 9/04; C12N 9/10; C12N 9/12; C12N 9/24; C12N 9/88; C12N 15/77; C12P 19/14

(52) U.S. Cl. CPC C12P 19/00; C12N 9/0006; C12N 9/1051; C12N 9/1205; C12N 9/2494; C12N 9/88; C12N 15/77; C12P 19/14; C12Y 101/01271; C12Y 204/01069; C12Y 302/01078; C12Y 402/01047; C12N 2800/101

(57) ABSTRACT

A genetically modified Corynebacterium for production of fucosyllactose, wherein the Corynebacterium has been modified to express a permease for lactose import, GDP-D-mannose-4,6-dehydratase (GMD), GDP-L-fucose synthase (WcaG) and fucosyltransferase (FucT) from exogenous nucleic acid sequences. The exogenous nucleic acid sequences encode a permease for lactose import and the GMD, WcaG and FucT are chromosomally integrated. The Corynebacterium additionally may comprise chromosomally integrated exogenous nucleic acid sequences for expression of phosphomannomutase (ManB) and GTP-mannose-1-phosphate guanylyltransferase (ManC). In certain embodiments, the Corynebacterium is Corynebacterium glutamicum. The Corynebacterium of the invention may be defective for functional expression of one or more glycosyltransferases involved in corynebacterial cell wall biosynthesis.

### CROSS-REFERENCE TO RELATED APPLICATIONS

This application is a U.S. National Stage Application based on International application No. PCT/EP2022/053368 filed Feb. 11, 2022 and claiming priority to European Patent application No. EP 21156553.6 filed Feb. 11, 2021.

### SEQUENCE LISTING

The instant application contains a Sequence Listing which has been submitted electronically in TXT format and is hereby incorporated by reference in its entirety. Said TXT copy, created on Mar. 27, 2024, is named 2397-20WO_Seq-Listing_011US1_ST25.txt and is 19,164 bytes in size.

### FIELD OF THE INVENTION

The invention relates to a genetically modified Corynebacterium for production of fucosyllactose, wherein the Corynebacterium has been modified to express a permease for lactose import, GDP-D-mannose-4,6-dehydratase (GMD), GDP-L-fucose synthase (WcaG) and fucosyltransferase (FucT) from exogenous nucleic acid sequences, characterized in that the exogenous nucleic acid sequences encoding a permease for lactose import, GMD, WcaG and FucT are chromosomally integrated. Preferably, the Corynebacterium additionally comprise chromosomally integrated exogenous nucleic acid sequences for expression of phosphomannomutase (ManB) and GTP-mannose-1-phosphate guanylyltransferase (ManC). In preferred embodiments, the Corynebacterium is Corynebacterium glutamicum. In embodiments, the Corynebacterium of the invention can be defective for functional expression of one or more glycosyltransferases involved in corynebacterial cell wall biosynthesis.

### BACKGROUND OF THE INVENTION

Human breast milk contains more than 200 structurally distinct oligosaccharides, hereinafter referred to as Human milk oligosaccharides (HMOs), with typical concentrations ranging between 5 to 25 g/l. Their composition and concentration vary during lactation and further depend on maternal genetics, diet, and health status. The presence of HMOs significantly mitigates acute infections in infants during/until the maturation of their immune system. Several HMOs used in clinical practice have proven to protect infants and adults from bacterial and viral infections. Additionally, some of the HMO constituents were reported to promote cognitive development and stimulate beneficial gut microbiota in infants. Hence, HMOs have gained increased attention in recent years as therapeutic agents and healthy supplements of human nutrition.

Fucosylated HMOs, in particular, α1,2-fucosyllactose (2FL) and α1,3-fucosyllactose (3FL) are dominant oligosaccharides in breast milk constituted in the range of 12-45% and 0.5-8% respectively. 2′-FL (Fucα1-2 [Galβ1-4]Glc) consists of L-fucose linked by an al-2 bond to the galactose residue in lactose, and its isomer 3-FL (Fucα1-3Galβ1-4Glc) consists of L-fucose linked by an al-3 bond to the glucose moiety in lactose. 2FL promotes early incidence of Bifidobacteria in gut microbiota and protects against diarrheal diseases caused by commensals in infants. it is known that about 20% of European and American women cannot synthesize 2FL due to the genetic mutation of fucosyltransferase genes. And in women who can produce 2FL, its concentration in milk decreases over the period of lactation. Hence, 2FL attracted much interest as a functional food ingredient in infant formula. For these reasons, there is a need to produce 2FL.

Fucosylated HMOs are either absent or present at a 100-to-1000-fold lower concentration in the milk of farm animals. The composition and variety of oligosaccharides from farm animals significantly differ from HMOs; furthermore, extraction from animal milk or whey is not a commercially viable option for largescale production. Chemical synthesis is challenging due to multistep synthesis and is accompanied by the cons of employing expensive substrates, low stereo-selectivity, production yield, and the use of toxic organic solvents. Biocatalytic synthesis is of limited applicability to scale-up due to the use of expensive substrates and unstable and low expression of fucosyltransferases. 2FL production using recombinant microorganisms subdue the drawbacks of chemical and enzymatic synthesis with the added advantage of scale-up and industrial production from inexpensive substrates.

Most conventional methods for producing 2′-fucosyllactose using microorganisms were produced using recombinant Escherichia coli. However, most Escherichia coli used for experimentation are predominantly known to be harmful to customers although they are not pathogens. Since an ingredient for the cell membrane of Escherichia coli may serve as endotoxin, high isolation, and purification (downstream processing) costs are involved in the production of 2′-fucosyllactose from Escherichia coli. Accordingly, there is a difficulty in using Escherichia coli as a host cell that produces fucosyllactose as food and medicinal materials ingredients.

To avoid the drawback of using the (potential) human pathogen E. coli for fucosyllactose production, US2020/048640A1 uses recombinant Corynebacterium glutamicum for producing fucosyllactose. However, the bacteria described therein contain the transgenes encoding for enzymes of the production pathway of fucosyllactose on plasmids that can easily be lost without constant antibiotic selection. Therefore, producing fucosyllactose employing this recombinant bacterium requires extensive downstream processing steps to purify 2FL. This is an important drawback of the described method since the produced 2FL can comprise antibiotics, which is a huge disadvantage for the intended subsequent use in baby food.

Because of the drawbacks of known methods and means for producing fucosyllactose, there remains a significant need in the art to provide additional means and methods for industrial production of fucosyllactose in a non-pathogenic and generally regarded as safe (GRAS) microorganism while avoiding the use of antibiotics during the production method.

Corynebacterium glutamicum has been industrially employed because of its natural competence to excrete glutamate under certain conditions. For example, biotin limitation, and/or by the addition of outer cell envelope-(inner cell membrane and cell wall) altering compounds such as penicillin and fatty acid ester surfactants which inhibit lipid synthesis.

Previous studies have used Corynebacterium glutamicum for the production of pigments, such as carotenoids by introducing synthetic operons into Corynebacterium glutamicum by chromosomal integration (Heider et al., Frontiers in Bioengineering and Biotechnology, 2014). The cell wall of Corynebacterium is multilayered and constitutes a barrier to many substances and compounds. The harvesting of the carotenoids in the prior art study of Heider was accomplished by batch cell harvest and extraction of the pigments from lysed Corynebacterium glutamicum cultures (Heider et al., 2014). This approach, however, is not applicable for large-scale nutrient production using Corynebacterium glutamicum, as such nutrients, e.g. hyaluronic acid, glutamate or sugars, are to be excreted in large amounts into the extracellular space to be harvested without the need to lyse the production strain. Unfortunately, the thick and impermeable cell wall of Corynebacterium glutamicum has been shown to be a limiting factor to the expression of large-scale nutrients.

For example, Corynebacterium was engineered for hyaluronic acid (HA) production. Unexpectedly the HA polymer, which was formed in the periplasmic region by plasma membrane bound enzyme HA synthase (HAS), was encapsulated in the inner membrane space of the cell, resulting in slow release of HA extracellularly and hence restricted nutrient uptake. The corynebacterial cell wall is thick and complex and the enzymes involved in its synthesis are not well characterized. Deleting the enzymes Cg0424 and Cg0420, which were suspected to have enzymatic activities as mannosyl-transferase and arabinosyl-transferase and catalyze the formation of mannan and arabinomannan on peptidoglycan layer, resulted in truncation of glycans in between outer and peptidoglycan layer and facilitated hyaluronidase to diffuse both the outer and middle peptidoglycan layer, resulting in the disruption of the encapsulation of the desired product and restoration of nutrient uptake (Wang Yang et al. 2020, “Eliminating the capsule-like layer to promote glucose uptake for hyaluronan production by engineered Corynebacterium glutamicum.” Nature communications, 11 (1) 3120).

However, production of desired compounds in Corynebacterium is not generally restricted or influenced by the cell wall and hence cannot generally be optimized by manipulation of the cell wall. In Corynebacterium that was engineered for the production of glutamate the manipulation of the cell membrane did not ensure or improve the stabilization of production. In said study extracellular glutamate excretion was limited despite the presence of mechanosensitive channel involved in the glutamate export (Ncg11221, cgp_1434) and also high metabolic flux towards glutamate production. (Hoischen C., et al., “Membrane alteration is necessary but not sufficient for effective glutamate secretion in Corynebacterium glutamicum.” J Bacteriol. 1990 June; 172 (6): 3409-16).

Other works to produce better strains to improve the glutamate export involved targeting enzymes involved in lipid and peptidoglycan synthesis were reported (Tuo et al., 2020, Biotechnology & Biotechnological Equipment, 34 (1), 38-47; Hirasawa T. et al., “A mutation in the Corynebacterium glutamicum ltsA gene causes susceptibility to lysozyme, temperature-sensitive growth, and L-glutamate production.” J Bacteriol. 2000; 182 (10): 2696-2701). Although Tuo et al. identified mutant genes associated with higher glutamate export by classical mutagenesis, a comparison of their catalytic activities with respect to wild-type enzymes was not performed. Furthermore, the transcriptomic changes at different growth temperatures of several species are well documented to vary. This may also be a contributing factor for glutamate excretion in addition to altered expression of the respective genes. Two of the enzymes Cg10688 and Cg10366 were essential components of the pyruvate dehydrogenase (PDH) that related to the acetyl-CoA synthesis participating at the pyruvate node. FabG and cg10286 are associated with fatty acid synthesis. The role of three genes cg2090, cg12139, and cg10628 remained unknown.

The products formed by the Corynebacterium are presumed to diffuse from inner plasma membrane to the fermentation media and levels are low even in the presence of an active transporter because of the presence of a mycolate layer with large amounts of non-covalently linked lipids and thick peptidoglycan layer. The mycolic acids are lipids linked through ester bonds to the arabinogalactan attached to the murein of the cell wall (Minnikin D. E. et al., 1987, “Chemical targets in cell envelopes”, p. 19-43; M. Hopper (ed.), Chemotherapy of tropical diseases. John Wiley & Sons Ltd., Chichester, UK). The chain lengths of these lipids in corynebacteria are of 22 to 38 carbon atoms (Yano et al., 1972, “Gas chromatographic and mass spectrometric analysis of molecular species of corynomycolic acids from Corynebacterium ulcerans.” FEBS Lett., 23:352-356).

Nevertheless, there are bottlenecks in identifying extra-cellular exporters of non-natural compounds produced by metabolic engineered Corynebacterium strains (Pérez-García F. et al., “Transport and metabolic engineering of the cell factory Corynebacterium glutamicum.”, FEMS Microbiol Lett. 2018; 365 (16)).

Hence, it is desirable to find new and reliable strategies to manipulate cellular export of the produced compounds. One strategy pursued herein was to target enzymes involved in the formation of different layers of cell wall. It was found that this may be a a fast and applicable strategy for developing viable industrial biotechnological processes.

Furthermore, the use of Corynebacterium glutamicum for producing biomolecules has been reported to be disadvantageous due to the strong tendency of the bacteria to produce foam during fermentation. In fact, agitation and aeration of Corynebacterium glutamicum cultures in a stirred tank bioreactor always cause foaming, and excess foaming forces the broth out of the bioreactor and contaminates the system. Accordingly, laborious optimization experiments must be performed to identify optimal aeration conditions that must be maintained to minimize foaming and maximize the specific production rate.

Accordingly, there is need in the art for means and methods that reduce foaming of Corynebacterium glutamicum in the context of the production of biomolecules such as fucosyllactose by these bacteria.

### SUMMARY OF THE INVENTION

Considering the prior art, the technical problem underlying the present invention is to provide alternative and/or improved means to produce fucosyllactose in transgenic bacteria. Furthermore, the provision of means and methods employing Corynebacterium glutamicum for producing biomolecules that reduce foam production by the bacterial cultures and therefore facilitate culture of these bacteria for industrial applications, ideally with increase production yields, are desired.

The problems underlying the present invention are solved by the features of the independent claims. Preferred embodiments of the present invention are provided by the dependent claims.

Aspects of the Invention Relating to Genetically Modified Corynebacterium with Chromosomally Integrated Transgenes Required for Production of Fucosyllactose

The invention therefore relates to genetically modified (or engineered) Corynebacterium for production of fucosyllactose, wherein the Corynebacterium has been modified to express a permease for lactose import, GDP-D-mannose-4,6-dehydratase (GMD), GDP-L-fucose synthase (WcaG) and fucosyltransferase (FucT) from exogenous nucleic acid sequences,

characterized in that the exogenous nucleic acid sequences encoding a permease for lactose import, GMD, WcaG and FucT are chromosomally integrated.

The development of the genetically modified Corynebacterium of the invention is highly advantageous in comparison to the state of the art. The bacteria of the invention enable highly effective production of fucosyllactose in industrial scale, for example for subsequent nutritional applications, in the absence of any antibiotics. Furthermore, the corynebacteria and in particular the preferred Corynebacterium glutamicum is not pathogenic in humans and has been considered as a generally recognized as safe (GRAS) host for the industrial production of biomolecules, such as in particular L-glutamate and L-lysine.

It is usually necessary to tune the expression levels of native and heterologous genes in microbial hosts to develop high-performance production strains. Most metabolic engineering efforts use multicopy plasmids because they offer an easy method for quickly prototyping different gene expression levels, but they have several drawbacks. Some cells might lose the plasmid because of its structural and segregational instability. Plasmid copy numbers are not constant for plasmids in stable populations. Additionally, selective pressure such as antibiotics is required to maintain the plasmids by the cell and may not always be effective in plasmid maintenance. Plasmids also put a metabolic burden on cells by diverting ribosomes and other cellular resources for their maintenance.

Importantly, most known processes for producing fucosyllactose, in GRAS hosts, have used the expression of plasmid-borne genes to express heterologous enzymes of the pathway in the host. Replicating plasmids are mostly useful for cloning and overexpression of recombinant genes, for the maximum overproduction of a given protein or for short-term lab scale synthesis of a product. However, for the in vivo synthesis of a non-natural product such as fucosyllactose in a bacterial host, where multiple genes must be expressed, the use of expression plasmids has several disadvantages. Different plasmids exhibit variable copy numbers based on the origin of replication, and consequently, differential expression of genes of the multi-enzymatic pathway results in flux imbalances thence poor product yields. Additionally, multicopy plasmids are segregationally unstable and detrimental to industrial-scale manufacturing processes. Particularly during high cell density fermentation and continuous fermentation conditions, there is the incidental loss of selective pressure due to reduced antibiotic activity because of degradation of antibiotics by the cell and culture dilution respectively. Auxotrophic strains although can maintain plasmids bearing necessary genes without antibiotics and selective pressure are also susceptible to plasmid loss due to leakage of the complementing product from the plasmid-bearing cell. Thus, the expression of the genes often fluctuates in the cell population, which results in population variation.

Moreover, the metabolic burden of plasmid replication and the strong overexpression of certain genes can lead to reduced growth rates and increased demand for energy and metabolites for the additional pathways. Structural instability of plasmids or transposon insertion in to expressing genes or gene loss due to recombination between multiple vectors lead to the takeover of culture by plasmids with inactivated genes.

Most importantly, in processes aimed at human nutrition, the use of antibiotics is either legally forbidden, not indicated or disadvantageous for safety reasons, or undesired for cost reasons. In addition, the for removal of antibiotics in downstream processing is elaborate and expensive.

To avoid these disadvantages, stable insertion of recombinant genes into the chromosome of the corynebacteria of the invention is performed. The chromosomal integration allows the stable maintenance of gene expression cassettes without the use of selection marker in strains.

Chromosomal integration and expression of genes is an effective alternative to construct stable and robust production strains and reduce leakiness of inducible expression systems, which are highly desirable traits for large-scale and long-term production processes. Despite the great potential of chromosome integration and the availability of genetic tools for targeted integration, achieving high gene expression levels from chromosomes remains far more challenging than manipulating expression using plasmids, even in well-studied bacterial species such as E. coli. In addition, there is evidence from studies in both prokaryotes and eukaryotes that chromosomal location can substantially affect heterologous gene expression levels.

Chromosomal position and spatial organization of gene cassettes are essential to maintain gene dosage and expression, but there is a lack of understanding of these positional effects in Corynebacterium and the prior art offers insufficient data how to mitigate the impact of expression silencing (Bryant, J. A. et al. “Chromosome position effects on gene expression in Escherichia coli K-12.” Nucleic Acids Res. 42, 11383-11392 (2014)).

Lange et al. 2017 attempted to identify chromosomal sites for gene integration, but positional effects at these sites remained unknown. A similar study expressing L-arabinose genes from heterologous weak Lac promotor in plasmid consumed 11 times more L-Arabinose (15.5 mM vs. 175 mM) in one-third the time (24 h vs. 8 hr) (Kawaguchi et al, 2008). Thus, the characterization of chromosomal location is essential for gene integration, discounting which may significantly reduce gene expression.

Further, while methods for chromosomal integration of expression cassettes are well established in E. coli such techniques have been proven difficult for performance in other bacteria and in particular in corynebacteria and Corynebacterium glutamicum.

Multiple factors regulate chromosomal gene expression. It is possible to manipulate the functional components of a genetic construct to optimize heterologous gene expression. These components include promoters, ribosome-binding sites, enhancers, and activators. Nevertheless, usually the expression of the same construct from a chromosome is weaker than from a plasmid. The location of gene integration is another factor that strongly influences gene expression from the chromosome and hence gene dosage. Depending on the growth phase of the cells, specific segments of the chromosome receive a higher gene dosage, which correlates with increased transcription of genes in those regions. Also, DNA compaction and proximity to active genes impact gene expression. Hence, modulation of gene integration positions is not trivial and random integration has a low chance of achieving the desired expression level. There has been no reliable method available in the prior art to predict optimal gene expression levels of individual genes required for any multi-gene biosynthetic pathways in microbial production systems.

Consequently, it is not possible to simply or routinely predict where a given biosynthetic pathway gene should be integrated into a chromosome. Furthermore, the expression of rate-limiting enzymes in the pathway is critical for production.

Herein, the inventors, however, evaluated the genomic regions of Corynebacterium with a reporter gene cassette and surprisingly found locations with minimal chromosomal effects, and herewith provide means for integrating gene cassettes in chromosomal regions of Corynebacterium resulting in unexpected beneficial effects on the production of fucosyllactose, as described herein in detail.

As shown in the examples below, it was surprisingly possible to efficiently construct genetically modified corynebacteria that comprise multiple expression cassettes (also called gene cassettes) for all required and advantageous genes of the fucosyllactose synthesis pathway. This was unexpected since the present invention required the integration of multiple expression cassettes into the bacterial chromosome. However, it is known that for Corynebacterium glutamicum genome modification by the integration of expression cassettes is laborious, time-consuming, and inefficient due to the rare event of genomic integration of the plasmid accompanied with screening several colonies after the second recombination to identify the desired recombination event. This process often becomes problematic when integrating multiple DNA fragments, especially if the expression cassettes have homologous sequences. The homologous sequences in different expression cassettes are problematic in generating strains because the cassettes may result in illegitimate recombination with each other. A homologous DNA segment as small as 8 to 12 base pairs can cause recombination in the genome (Mateos et al., 1996, J Bacteriol., 178 (19): 5768-75). Hence it is a limitation to integrate multiple copies of the same gene in the chromosome which is an important prerequisite to regulate the expression of multiple genes in the pathway.

In the context of the present invention, the difficulty of integrating multiple DNA fragments and avoided illegitimate recombination could be overcome by using expression cassettes with heterologous sequences encoding the respective gene functions from different organisms and by minimizing multiple usage of promotor and terminator sequences. Furthermore, insertion sites are chosen in between essential genes on the chromosomes to subdue recombination between expression cassettes with homologous sequences.

Accordingly, in embodiments, the chromosomally integrated exogenous nucleic acid sequences encoding a permease for lactose import, GMD, WcaG and FucT comprise multiple expression cassettes which are integrated in different chromosomal intergenic regions. Preferably, the integration/insertion sites of the cassettes are located in different intergenic regions between different essential genes of the bacteria.

In embodiments, the genetically modified bacteria of the invention comprise 3, 4, 5, 6, 7, 8, 9 or 10 expression cassettes. Preferably, the genetically modified bacteria of the invention comprise at least 5, 6 or 8 expression cassettes.

In embodiments, each expression cassette of the transgenic bacterium of the invention has a different promoter sequence. In preferred embodiments, no more than two of the expression cassettes of the transgenic bacteria comprise the same promoter. In embodiments, no more than three of the expression cassettes of the transgenic bacteria comprise the same promoter. In embodiments where two or more expression cassettes comprise the same promoter, it is preferred that the promoter present in two or more of the expression cassettes is the Tuf and/or Tac promoter.

In embodiments, each expression cassette of the transgenic bacterium of the invention has a different terminator sequence. In preferred embodiments, no more than two of the expression cassettes of the transgenic bacteria comprise the same terminator. In embodiments, no more than three of the expression cassettes of the transgenic bacteria comprise the same terminator.

In embodiments, the expression cassettes comprise one or more heterologous promoters. In embodiments, the expression cassettes comprise one or more corynebacterial promoters. In embodiments, the expression cassettes comprise one or more heterologous promoters and one or more corynebacterial promoters.

In embodiments comprising corynebacterial promoters, such as Tuf and/or GlyA promoters from Corynebacterium, the expression cassettes comprising corynebacterial promoters are integrated at intergenic sites spanning essential genes.

Additionally, there is a need for high-level expression of selective pathway genes to improve flux towards fucosyllactose production from glycolytic intermediate Fructose-6-phosphate without impairing cell growth, which needs testing both constitutive and regulated promotors. Gene dosage and promotor strength are important considerations during strain construction process.

In the context of the present invention, high expression of all transgenes of the fucosyllactose production pathways could be ensured by using a specific selection of inducible promoters for expression of regulatory genes in the pathway especially GMD and ManB. For example, in the preferred example MB02 described herein, ManB_ManC and GMD_WcaG operons are subjected to inducible expression. It can also be preferred that FucT is under inducible expression, as described herein for HpFucT in the base strain MB002. To achieve inducible expression of the pathway genes, a gene cassette of Lactose repressor gene LacI driven from a LacIq promotor is introduced in the chromosome additionally.

Further improvement in 2FL production can be attained with constitutive expression of additional genes. In embodiments, a WcaG-GMD operon is under the control of a constitutive promotor, such as a Tuf promoter, with Lac Operator to effectuate IPTG inducible expression to avoid growth impairment during early and log phase.

Mobile genetic elements such as transposase genes are detrimental to heterologous protein expression (Choi, Jae Woong, et al. “Enhanced Production of Recombinant Proteins with Corynebacterium glutamicum by Deletion of Insertion Sequences (IS Elements).” Microbial Cell Factories, vol. 14, no. 1, 2015, pp. 207-207).

In preferred embodiments of the invention, suitable chromosomal integration sites for the exogenous nucleic acid sequences (transgenes/expression cassettes) were selected to integrate gene cassettes with concomitant deletion of transposase gene. Reproducible gene expression from selected sites is determined based on relative expression of an integrated reporter gene cassette, such as preferably the E. coli galactosidase (LacZ) gene under the control of constitutive LacuV5 promotor. Sites with high LacZ expression levels are selected further to integrate 2FL pathway gene cassettes. Accordingly, in embodiments the integration sites of the exogenous nucleic acid sequences of genetically modified Corynebacterium have been selected based on suitability for exogenous gene expression as assessed using a reporter gene expression cassette, and preferably also suitability for integration of 2FL pathway gene cassettes.

Preferred chromosomal integration sites for exogenous nucleic acid sequences of the invention have been disclosed in the examples below. In the context of the present invention, preferred integration sites for expression cassettes comprise integrations leading to deletion of following transposase genes: Δcgp_2725, Δcgp_1213, Δcgp_2854, Δcgp_3151, Δcgp_1178, Δcgp_1782, Δcgp_2600.

The surprising increase in reporter gene expression in the preferred sites for integration in Corynebacterium is shown in FIG. 7 and is discussed in the example below.

As used herein, the term genetically modified Corynebacterium and genetically engineered Corynebacterium are used interchangeably.

Besides the provision of exogenous fucosyltransferase (FucT), Corynebacterium requires incorporation of GDP-D-mannose-4,6-dehydratase (Gmd), GDP-L-fucose synthase (this enzyme is also called “GDP-4-keto-6-deoxy-D-mannose-3,5-epimerase-4-reductase” and is also simply referred to as “WcaG”), and a permease for lactose import, such as lactose permease (LacY).

Importantly, in contrast to approaches for fucosyllactose production using E. coli, where E. coli has genes encoding Gmd, WcaG, and lactose permease (LacY), Corynebacterium glutamicum has no genes encoding these enzymes, so provision of external coding nucleic acid sequences for expression of these genes is required.

In embodiments, genes encoding α-1,2-fucosyltransferase are derived from Helicobacter pylori, and genes encoding Gmd, WcaG and lactose permease (LacY) are derived from Escherichia coli.

In embodiments, the Corynebacterium additionally comprise chromosomally integrated exogenous nucleic acid sequences for expression of phosphomannomutase (ManB) and GTP-mannose-1-phosphate guanylyltransferase (ManC).

Corynebacterium glutamicum possesses genes encoding phosphomannomutase (ManB) and GTP-mannose-1-phosphate guanylyltransferase (ManC) and can thus express the same. Therefore, it may not be absolutely required that these genes are further expressed from exogenous nucleic acid sequence. However, increased expression of these enzymes is advantageous for mass-production of fucosyllactose and for this reason, the present invention preferably comprises chromosomally integrated exogenous nucleic acid sequences for expression of ManB and ManC.

In preferred embodiments, the Corynebacterium additionally comprise a chromosomally integrated exogenous nucleic acid sequence, such as an expression cassette, for expression of a lactose repressor. A lactose repressor (or lac repressor; encoded by a lactose repressor gene) is a DNA-binding protein that inhibits the expression of transgenes of the invention present in an expression cassette comprising the lac operon or parts thereof in the absence of lactose or IPTG. Corresponding inducible genetic systems for inducible transgene expression are known in the art. In the context of the invention, Lac repressor controlled inducible expression of heterologous genes can comprise the use of Lac/IPTG inducible promotors such as Lac, Tac, Tre etc. In embodiments, synthetic chemical IPTG can also be used for induction of respective genes.

In the context of the present invention, lactose is provided for the synthesis of FL by the transgenic bacteria of the invention. It is advantageous to include a transgenically expressed lac repressor in the bacteria of the invention. Suitable lac repressors of the invention include LacI gene from E. coli.

In embodiments, expression cassettes of the invention comprise a promoter and a coding sequence. In embodiments, expression cassettes of the invention comprise a promoter, an operator and a coding sequence. In embodiments, expression cassettes of the invention comprise a promoter, an operator, a terminator sequence and a coding sequence. In embodiments, expression cassettes of the invention comprise a promoter, an operator, a terminator sequence and/or a coding sequence. In embodiments, such expression cassettes may comprise more than one coding sequence.

Preferred promoters to be used in the context of the invention comprise Tac promoter, Tuf promoter, GlyA promoter, Lac promoter and Tre promoter. Preferred operators of the invention comprise the lac operator. Preferred terminators comprise T7 terminator and rmB terminator.

Preferred promoters to be used for the transgenes of the invention have been described in the examples below. The individual expression cassettes described therein, including cassettes for single genes or cassettes comprising more than one gene, such as the operons described therein, can be used independently in the context of the invention have not to be used in the exact combination disclosed in the examples below, as is understood by the skilled person.

In embodiments of the genetically modified Corynebacterium for production of fucosyllactose, ManC and ManB are encoded by a continuous exogenous nucleic acid sequence comprising coding sequences for ManC and ManB within a single operon. Such an operon may be controlled by a single promoter, such as a Tac promoter.

In embodiments, ManC and ManB are encoded by separate (non-continuous) exogenous nucleic acid sequence and preferably comprise independent regulatory sequences. The respective coding sequences can be integrated at independent locations within the Corynebacterium chromosome.

As disclosed herein, the corynebacteria of the invention can comprise each transgene separately, e.g. in a separate exogenous nucleic acid sequence preferably with independent regulatory sequences. In alternative embodiments, two or more of the transgenes of the corynebacteria of the invention can be comprised/encoded by a continuous exogenous nucleic acid sequence comprising coding sequences of the two or more transgenes within a single operon.

Each of the transgenes of the Corynebacterium of the invention can be present in more than one copy, such as 2, 3, 4, 5, 6, 7, 8, 9 or 10 copies, wherein the number of copies of one transgene can be independent of the number of copies of the other transgenes. Therein, the copies of one transgene can be of the same (identical coding sequences of the respective transgene) or different variants of the transgene, such as transgenes of different origins. For example, in case of FucT, the bacteria of the invention can comprise two exogenous nucleic acid sequences encoding FucT (two copies), wherein one copy can be, for example, a FucT encoding sequence from Helicobacter pylori, and the other copy can be, for example, a FucT encoding sequence from Helicobacter mustelae. The same holds true for each of the different transgenes of the Corynebacterium of the invention.

In preferred embodiments, the Corynebacterium is Corynebacterium glutamicum.

Corynebacterium glutamicum is a frequently used and important microorganisms in white biotechnology, due to its classification as a GRAS host.

In embodiments, the genetically modified Corynebacterium of the invention exports fucosyllactose.

In embodiments of the invention, the genetically modified Corynebacterium is engineered to export fucosyllactose into the medium when grown at appropriate culture conditions. It is understood that export of FL relates to any kind of process, active or passive, that leads to FL being present outside the bacteria in the culture medium.

In embodiments, the FucT comprised by the genetically engineered Corynebacterium of the invention is alpha-1,2-FucT. In further embodiments, the FucT comprises alpha-1,2-FucT.

To produce 2′-FL, the use of a suitable alpha-1,2-FucT is preferred. Corynebacteria comprising alpha-1,2-FucT from Helicobacter pylori are particularly preferred. Alternatively, or in addition, alpha 1,2FucT WbgL from E. coli 0126 or Thermosynechococcus elongatus may be used. Alternatively, or in addition, the alpha-1,2-FucT from Helicobacter mustelae may be used.

In embodiments, the permease for lactose import comprised by the genetically engineered Corynebacterium of the invention is a lactose permease (LacY).

In embodiments, the Corynebacterium comprises at least one exogenous nucleic acid sequences encoding FucT, wherein a FucT coding sequence is from Helicobacter pylori.

In embodiments, the Corynebacterium comprises at least one exogenous nucleic acid sequences encoding FucT, wherein expression of a FucT coding sequence is controlled by a Tre promoter.

In embodiments, the Corynebacterium comprises at least one exogenous nucleic acid sequences encoding FucT, wherein a FucT coding sequence is from Helicobacter pylori and expression of a FucT coding sequence is controlled by a Tre promoter.

In embodiments, the Corynebacterium comprises at least one exogenous nucleic acid sequences encoding FucT, wherein a FucT coding sequence is codon optimized from Helicobacter mustelae.

In embodiments, the Corynebacterium comprises at least one exogenous nucleic acid sequences encoding FucT, wherein expression of a FucT coding sequence is controlled by a Tuf promoter.

In embodiments, the Corynebacterium comprises at least one exogenous nucleic acid sequences encoding FucT, wherein a FucT coding sequence is codon optimized from Helicobacter mustelae and expression of a FucT coding sequence is controlled by a Tuf promoter.

In embodiments, the Corynebacterium comprises at least one exogenous nucleic acid sequences encoding FucT, wherein a FucT coding sequence is from Helicobacter pylori and expression of a FucT coding sequence is controlled by a Tuf promoter.

In embodiments, the Corynebacterium comprises at least one exogenous nucleic acid sequences encoding FucT, wherein a FucT coding sequence is codon optimized from Helicobacter mustelae and expression of a FucT coding sequence is controlled by a Tre promoter.

In embodiments, the Corynebacterium comprises at least two exogenous nucleic acid sequences encoding FucT.

In embodiments, the Corynebacterium comprises at least two exogenous nucleic acid sequences encoding FucT, wherein preferably
i. a first FucT coding sequence is from Helicobacter pylori and/or expression of a first FucT coding sequence is controlled by a Tre promoter, and
ii. a second FucT coding sequence is codon optimized from Helicobacter mustelae and/or expression of a second FucT coding sequence is controlled by a Tuf promoter.

In embodiments of the Corynebacterium of the invention, the Corynebacterium comprises at least two exogenous nucleic acid sequences encoding FucT.

In embodiments, the Corynebacterium of the invention comprises a FucT from Helicobacter pylori.

In embodiments, the Corynebacterium of the invention comprises a FucT coding sequence controlled by a Tre promoter.

In embodiments, the Corynebacterium of the invention comprises a FucT from Helicobacter mustelae.

In embodiments, the Corynebacterium of the invention comprises a FucT coding sequence controlled by a Tuf promoter.

In embodiments, the FucT comprised by the Corynebacterium of the invention comprises a FucT from Helicobacter pylori and a FucT from Helicobacter mustelae.

In preferred embodiments of the genetically modified Corynebacterium of the invention, WcaG and GMD are encoded by a continuous exogenous nucleic acid sequence comprising coding sequences for WcaG and GMD within a single operon.

In embodiments, WcaG and GMD are encoded by separate (non-continuous) exogenous nucleic acid sequence and preferably comprise independent regulatory sequences. The respective coding sequences can be integrated at independent locations within the Corynebacterium chromosome.

In embodiments, the Corynebacterium of the invention comprises at least one exogenous nucleic acid sequence encoding WcaG and GMD are encoded as a single fusion gene expressing both the activities of GMD and WcaG.

In embodiments, the coding sequences for WcaG and GMD are from Escherichia coli.

In embodiments, the genetically modified Corynebacterium of the invention comprises at least one exogenous nucleic acid sequences encoding WcaG and GMD in a continuous exogenous nucleic acid sequence within a single operon. In embodiments, a WcaG and GMD encoding operon is controlled by a Tac promoter. In embodiments, the coding sequences of WcaG and/or GMD are from Escherichia coli. In embodiments, a WcaG and GMD encoding operon is controlled by a Tuf promoter. In embodiments, the coding sequences of WcaG and/or GMD are from Bacteroides fragilis.

In embodiments, the genetically modified Corynebacterium of the invention comprises at least two exogenous nucleic acid sequences encoding WcaG and GMD in a continuous exogenous nucleic acid sequence within a single operon, wherein preferably
i. a first WcaG and GMD encoding operon is controlled by a Tac promoter and/or the coding sequences are from Escherichia coli, and
ii. a second WcaG and GMD encoding operon is controlled by a Tuf promoter and/or the coding sequences are from Bacteroides fragilis.

In embodiments, the genetically modified Corynebacterium of the invention comprises at least one exogenous nucleic acid sequence encoding a permease for lactose import.

In embodiments, expression of a permease for lactose import coding sequence is controlled by a gly A promoter.

In embodiments, a permease for lactose import is LacY from Lactobacillus delbrueckii.

In embodiments, expression of a permease for lactose import coding sequence is controlled by a constitutive Lac promoter. In embodiments, a permease for lactose import is LacY from Escherichia coli.

In embodiments, the genetically modified Corynebacterium of the invention comprises at least two exogenous nucleic acid sequences encoding a permease for lactose import, wherein preferably
i. expression of a first permease for lactose import coding sequence is controlled by a gly A promoter and/or a first permease for lactose import coding sequence is LacY from Lactobacillus delbrueckii, and
ii. expression of a second permease for lactose import coding sequence is controlled by a constitutive Lac promoter and/or is LacY from Escherichia coli.

In preferred embodiments, the Corynebacterium is Corynebacterium glutamicum and comprises
at least two exogenous nucleic acid sequences encoding FucT,
at least two exogenous nucleic acid sequences encoding WcaG
at least two exogenous nucleic acid sequences encoding GMD, and
at least two exogenous nucleic acid sequences encoding LacY.

In such embodiments, exogenous nucleic acid sequences preferably comprise coding sequences for WcaG and GMD in a continuous nucleic acid sequence within a single operon. In preferred embodiments, corynebacteria of the invention comprise two (or more) such operons with coding sequence for WcaG and GMD.

In preferred embodiments, the Corynebacterium is Corynebacterium glutamicum and comprises
at least two exogenous nucleic acid sequences encoding FucT,
at least two exogenous nucleic acid sequences encoding WcaG and GMD in a continuous exogenous nucleic acid sequence within a single operon, and
at least two exogenous nucleic acid sequences encoding LacY.

Herein the inventors found the presently used method of integrating genes into Corynebacterium to be a fast, safe and applicable strategy in comparison to classical mutagenesis. Cellular export of the produced compounds is desirable for developing viable industrial biotechnological processes. Herein it was found that targeting enzymes involved in the formation of the cell wall facilitates fast diffusion of products from cytoplasm to fermentation broth. The inventors herein systematically deleted enzymes that are involved in the synthesis of three layers of corynebacterial cell wall in a 2FL producing strain and identified enzymes that when deleted surprisingly increased 2FL production.

Cell walls of Corynebacterium are intricate and multilayered. The cytoplasmic or plasma membrane is lined with glycolipids bound to a thick peptidoglycan layer. These layers protect the cell from various stress conditions. The mycolic acids of the outer lipid-rich layer are covalently linked to the peptidoglycan surface by a polysaccharide mesh of arabinogalactans. Consequently, in the absence of a specific transporter or porin, these multiple layers impede metabolite diffusion from the cytoplasm to the surrounding environment and vice versa.

Glucopyranosyluronic acid diacylglycerol (Gl-A), phosphatidyl-myo-inositol mannosides (PIMs), and phosphatidylglycerol (PG) are structural phospholipids of the cytoplasmic membrane. Membrane PIMs and GI-As serve as attachment sites for polymeric mannose, which is conjugated to GT-C glycosyltransferases and forms mature lipomannans (LMs) and lipoarabinomannans (LAMs). Cgp_2400 and cgp_1876 encode glycosyltransferases that produce PIMs and cgp_0554 catalyzes Gl-A respectively.

Polyprenol monophosphomannose (PPM) is the periplasmic mannosyl donor for both lipoglycan biosynthesis and protein glycosylation and is synthesized in the cytosol by the glycosyltransferase family member cgp_1672, a polyprenol-monophosphomannose synthase (Mohiman N, et al. PloS One, 2012). The ppm operon is essential for acylation and glycosylation of lipoproteins in Corynebacterium glutamicum. PPM translocated to the periplasmic face of the inner membrane is a substrate for the glycosyltransferase family members cgp_2385, egp_3164, cgp_2393, and cgp_2390. In LMs and LAMs, cgp_2385 and cgp_3164 catalyze the formation of a longer (1→6) mannan backbone. Their deletion results in short mannans, whereas CGP_2393 and cgp_2390 encode (1→2)-mannopyranosyltransferases which are a requisite for the synthesis of the a (1→2) linkages in the mannan backbones of LMs and LAMs. A variety of arabinofuranosyl-transferases carry out a stepwise assembly of the branched domains found in lipoarabinomannan (LAM) and arabinogalactan (AG), which are the two largest cell wall lipopolysaccharides. cgp_2389 encode α(1→2) arabinofuranosyltransferase, biosynthesis of singular arabinans of LAM.

Peptidoglycan (PG) is synthesized on the outer surface of the cytoplasmic membrane to maintain the structural integrity of the cell. PG is a strong scaffold-like structure, consisting of polymeric glycan strands cross-linked by short peptides, resulting in a homogenous molecule that encloses the cell. PG synthesis is catalyzed by membrane-bound enzymes called penicillin-binding proteins (PBPs). They are classified based on the enzymatic domains that catalyze transglycosylation (formation of the glycan strands from the disaccharide) and transpeptidation (cross-linking the glycan strands through the peptide portion). There are three classes of PBPs: class A high-molecular-weight PBPs (HMW-PBPs), with both transpeptidase and transglycosylase domains; class B HMW-PBPs, with an N-terminal domain of the unknown function and a transpeptidase domain; and low-molecular-weight PBPs (LMW-PBPs), with d,d-carboxypeptidase, or endopeptidase activity (Valbuena N, et al., Mol Microbiol. 2007, “Characterization of HMW-PBPs from the rod-shaped actinomycete Corynebacterium glutamicum: peptidoglycan synthesis in cells lacking actin-like cytoskeletal structures”; Mol Microbiol. 2007 November; 66 (3): 643-57.).

A mutant and deletion of a homolog of cgp_3313 in strain ATCC 13869 enhanced the transformation efficiency by 19.25-fold and 4.89-fold respectively in the absence of cell wall weakening agents. However, it did not result in enhanced glutamate production or excretion (Liu J, et al., “Mutations in Peptidoglycan Synthesis Gene ponA Improve Electrotransformation Efficiency of Corynebacterium glutamicum ATCC 13869” Appl Environ Microbiol. 2018 Nov. 30; 84 (24): e02225-18).

The inventors herein deleted both cgp_3313 and its close homolog of cgp_0336 in strain MB007 to test the export of 2FL and observed the surprising effect of improvement of 2FL production and improvement of 2FL excretion/efflux (see FIG. 9).

In embodiments, the genetically modified Corynebacterium of the invention is defective for functional expression of one or more glycosyltransferases involved in corynebacterial cell wall biosynthesis.

Surprisingly, it was found out that deficiency in glycosyltransferases involved in corynebacterial cell wall biosynthesis does not affect the efficiency of intracellular metabolic pathways and in particular not of an introduced fucosyllactose biosynthesis pathway.

Importantly, however, such a functional deficiency that can be caused by inactivation of one or more of such glycosyltransferases reduce the foam production by corynebacterial cultures during production processes and therefore make such processes much easier to handle. Furthermore, it is possible to scale up the volumes of the bacterial cultures, since no additional space in the culture vessels/containers has be reserved for foam that is produced during the culture process.

Unexpectedly, the reduction of foam does not only lead to such practical advantages for the culture and production process, but it turned out that deficiency for one or more glycosyltransferases involved in corynebacterial cell wall biosynthesis also allows a more efficient production and purification of fucosyllactose from the bacterial culture. Therefore, besides and independent from the possibility of culturing larger volumes, it is also possible to purify higher amounts of fucosyllactose from identical culture volumes in comparison to produce corynebacteria that have are not defective for one or more glycosyltransferases involved in corynebacterial cell wall biosynthesis.

Therein, the one or more glycosyltransferases are selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

Therein, the one or more glycosyltransferases are selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

Therein, the one or more glycosyltransferases are preferably selected from the group comprising or consisting of cgp_3313 (MreB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_2393 (GT87,GT87); cgp_2390 (GT87); cgp_2389 (GT87); cgp_2385 (GT87); and cgp_3164.

In embodiments, Corynebacterium is defective for functional expression of one glycosyltransferase involved in corynebacterial cell wall biosynthesis selected from the group comprising or consisting of egp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments, Corynebacterium is defective for functional expression of one glycosyltransferase involved in corynebacterial cell wall biosynthesis selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_1180 (GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments, Corynebacterium is defective for functional expression of one glycosyltransferase involved in corynebacterial cell wall biosynthesis selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_2393 (GT87,GT87); cgp_2390 (GT87); cgp_2389 (GT87); cgp_2385 (GT87); and cgp_3164.

In embodiments, the one or more glycosyltransferases are selected from the group comprising or consisting of cgp_0336 (GT51), cgp_2385 (GT87), cgp_1672 (PpmC; GT2); cgp_2390 (GT87), cgp_2393 (GT87), cgp_0554 (GT4), cgp_1876 (GT4), cgp_1877 (GT4), cgp_2400 (GT4) and cgp_3191 (GT2).

In preferred embodiments, the corynebacteria of the invention are defective for cgp_3164.

In preferred embodiments, the corynebacteria of the invention are defective for cgp_0554.

In preferred embodiments, the corynebacteria of the invention are defective for cgp_2385.

In preferred embodiments, the corynebacteria of the invention are defective for cgp_3191.

In preferred embodiments, the corynebacteria of the invention are defective for cgp_0336.

In preferred embodiments, the one or more glycosyltransferases are selected from the group comprising or consisting of cgp_0336 (GT51), cgp_2385 (GT87), cgp_0554 (GT4), cgp_3164 and cgp_3191 (GT2).

In a further aspect, the invention relates to a method for producing fucosyllactose, the method comprising culturing a genetically modified Corynebacterium of the present invention comprising the transgenes for fucosyllactose production as described herein in a medium supplemented with lactose.

In embodiments of the method of the invention, the culture medium additionally comprises glucose.

Preferably, the culture medium of the present invention does not comprise antibiotics.

Any features of the invention described in the context of the bacteria of the invention are herewith also disclosed in the context of the method of the invention and the other way around.

Aspects of the Invention Relating to Genetically Modified Corynebacterium with One or More Defective Glycosyltransferases Involved in Corynebacterial Cell Wall Biosynthesis

In a further aspect, the invention relates to a genetically modified (or engineered) Corynebacterium for production of biomolecules, characterized in that the Corynebacterium is defective for functional expression of one or more glycosyltransferases involved in corynebacterial cell wall biosynthesis.

Surprisingly, it was found out that deficiency in glycosyltransferases involved in corynebacterial cell wall biosynthesis does not significantly impair the growth and efficiency of intracellular metabolic pathways, in particular not of an introduced fucosyllactose biosynthesis pathway.

Corynebacterial cell wall is multilayered, the phospholipidic cytoplasmic membrane is surrounded by a thick arabinogalactan-peptidoglycan sheath covalently attached to an outer membrane called mycomembrane. The mycomembrane is a complex ensemble of mycolic acids, phospholipids, lipoglycans and proteins forming a hydrophobic bilayer. It is a major permeability barrier for the transport of small hydrophilic solutes and antimicrobial compounds in genus Corynebacteria.

When testing FL production in Corynebacterium, it was found that efflux of 2FL in Corynebacterium is hampered significantly by the bacterial cell wall. Whole cell strain producing FL intracellularly in higher quantities results in accumulation of metabolic intermediates known to inhibit rate limiting genes such as ManB and GMD of the pathway. This might lead to metabolic burden on the cell and thus growth arrest or inefficient synthesis of FL. Such Inefficient fermentation and recovery processes consequently increase economic costs to scale up. Thus, it was investigated whether membrane fluidity could be improved by systematically deleting glycosyltransferases involved in the formation of the several layers of the Corynebacterial cell wall, and whether such deletions have an effect on efflux of biomolecules such as Fucosyllactose from these genetically modified Corynebacteria.

Importantly, however, it was surprisingly found that such a functional deficiency that can be caused by inactivation of one or more glycosyltransferases involved in corynebacterial cell wall biosynthesis reduces the foam production of corynebacterial cultures during production processes and therefore make such processes much easier to handle. Therefore, it is also possible to scale up the volumes of the bacterial cultures, since no additional space in the culture vessels/containers has be reserved for foam that is produced during the culture process. Also, foaming can lead to contamination and loss of material, either of which will reduce yields.

Foaming is a serious problem in corynebacterial fermentations, and a critical factor to be monitored and controlled by adding antifoam agents manually in lab scale bioprocess experiments. Process optimization accompany controlling both aeration and agitation to minimize foaming. Agitation and aeration in a stirred tank bioreactor always cause foaming, uncontrolled foaming resulting in the loss of aseptic conditions; the fermenter becomes contaminated and microorganisms are released into the environment. Additionally, foam formation may cause serious operational difficulties by blocking air filters in aerated stirred tank bioreactors, especially in high cell density cultivations. Foam formation is generally attributed to the increase in viscosity of the media due to increase in denatured proteins/peptides and surface-active agents formed during culture process and cell lysis at the air liquid interface. However additional factors might contribute in corynebacterial bioprocess with its outer membrane significantly composed of hydrophobic and amphiphilic constituents such as mycolic acids and phospholipids.

Furthermore, reduced foaming facilitates efficient oxygen exchange during fermentation. Growth of aerobic organisms require a sufficient concentration of dissolved oxygen in the medium, measured as volume of dissolved oxygen coefficient (KLa). The kLa is a measure of how much oxygen is transferred into the medium over a certain amount of time and it is not improved with increase in aeration in corynebacterial cultures, because kLa of a system can be influenced by several factors such as properties of the medium like viscosity, the presence of organisms and their by-products, excess addition of antifoam agents (Çalιk G, Ünlütabak F, Özdamar T H. Product and by-product distributions in glutamic acid fermentation by Brevibacterium flavum: effects of the oxygen transfer. J Biochem Eng J. 2001; 9:91-101; Stanbury, P. F., Whitaker, A., & Hall, S. J. (2013). Principles of fermentation technology. Elsevier). Foaming significantly reduce kLa, and oxygen transfer rate (OTR) depends upon the kLa, (Routledge, S. J. (2012). Beyond de-foaming: the effects of antifoams on bioprocess productivity. Comput. Struct. Biotechnol. J. 3. doi:10.5936/csbj.201210014). Foaming also produce more waste and reduce the amount of medium and carbon sources available for bacterial growth.

There are basically three methods used to control foam production: media modification, mechanical foam-breaking devices, or the automatic addition of chemical antifoam agents. If foaming is minimized, then throughputs can be increased. Various procedures have been used in industry to reduce foam formation rate, with each of them having its own advantages and disadvantages.

Strain improvement is a vital part of process development in most fermentation industries. It provides a means by which production costs can be reduced through increases in productivity by optimal utilization of carbon sources and reduction of manufacturing costs. As described herein, surprisingly such desirable characteristics, in particular reduced foaming of Corynebacterial cultures, were identified while constructing the genetically modified strains with the aim of improving 2FL efflux.

This is advantageous not only in the context of fucosyllactose production as described herein. In contrast, these advantages of this aspect of the invention can also be harnessed for the production of other biomolecules by corynebacteria and in particular by Corynebacterium glutamicum, since the advantages of defects in cell wall biosynthesis are universal to such bacterial cultures and production of biomolecules by such cultures, such as for example L-glutamate and L-lysine, lactate, cadaverine, isobutanol, succinate, itaconate, plant-derived polyphenols.

Unexpectedly, the reduction of foam does not only lead to such practical advantages for the culture and production process (see reduced foaming in fed-batch of strain according to the invention in FIG. 11), but it turned out that deficiency for one or more glycosyltransferases involved in corynebacterial cell wall biosynthesis also allows a more efficient production and purification of biomolecules by the bacteria, as exemplified for fucosyllactose in the examples below. Therefore, besides and independent from the possibility of culturing larger volumes, it is also possible to purify higher amounts of fucosyllactose from identical culture volumes in comparison to producing corynebacteria that are not defective for one or more glycosyltransferases involved in corynebacterial cell wall biosynthesis.

Therein, the one or more glycosyltransferases are selected from the group comprising cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

Therein, the one or more glycosyltransferases are selected from the group comprising cgp_3313 (MreB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_1180 (GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

Or therein, the one or more glycosyltransferases are preferably selected from the group comprising cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_2393 (GT87,GT87); cgp_2390 (GT87); cgp_2389 (GT87); egp_2385 (GT87); and cgp_3164.

In embodiments, Corynebacterium is defective for functional expression of one glycosyltransferase involved in corynebacterial cell wall biosynthesis selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments, Corynebacterium is defective for functional expression of one glycosyltransferase involved in corynebacterial cell wall biosynthesis selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments, Corynebacterium is defective for functional expression of one glycosyltransferase involved in corynebacterial cell wall biosynthesis preferably selected from the group comprising or consisting of cgp_3313 (MreB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_2393 (GT87,GT87); cgp_2390 (GT87); cgp_2389 (GT87); egp_2385 (GT87); and cgp_3164.

In embodiments, the one or more glycosyltransferases are selected from the group comprising or consisting of cgp_0336 (GT51), cgp_2385 (GT87), cgp_1672 (PpmC; GT2); cgp_2390 (GT87), cgp_2393 (GT87), cgp_0554 (GT4), cgp_1876 (GT4), cgp_1877 (GT4), cgp_2400 (GT4) and cgp_3191 (GT2).

In preferred embodiments, the corynebacteria of the invention are defective for cgp_3164.

In preferred embodiments, the corynebacteria of the invention are defective for cgp_0554.

In preferred embodiments, the corynebacteria of the invention are defective for cgp_2385.

In preferred embodiments, the corynebacteria of the invention are defective for cgp_3191.

In preferred embodiments, the corynebacteria of the invention are defective for cgp_0336.

In preferred embodiments, the one or more glycosyltransferases are selected from the group comprising or consisting of cgp_0336 (GT51), cgp_2385 (GT87), cgp_0554 (GT4), cgp_3164 and cgp_3191 (GT2).

In embodiments of the invention, the one or more glycosyltransferases are selected from the group comprising or consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments of the invention, the one or more glycosyltransferases are selected from the group comprising or consisting of cgp_3313 (MrcB; GT51), cgp_0336 (PonA; GT51); cgp_3166 (GT4); cgp_2400 (GT4); cgp_1876 (GT4); egp_1268 (GlgA; GT4); cgp_0554 (GT4); cgp_3191 (GlfT; GT2); cgp_1672 (PpmC; GT2); cgp_1180 (GT2); cgp_0730 (GT2); cgp_0396 (GT2); cgp_0394 (GT2); cgp_0246 (GT2); egp_0163 (GT2); cgp_2393 (GT87,GT87); egp_2390 (GT87); cgp_2389 (GT87); cgp_2385 (GT87); and cgp_3164.

In embodiments of the invention, the one or more glycosyltransferases are preferably selected from the group comprising or consisting of egp_3313 (MrcB; GT51), egp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_2393 (GT87,GT87); cgp_2390 (GT87); cgp_2389 (GT87); cgp_2385 (GT87); and cgp_3164.

In embodiments, the one or more glycosyltransferases are selected from the group comprising or consisting of cg0336 (GT51), cg2385 (GT87), cgp_1672 (PpmC; GT2); cg2390 (GT87), cg2393 (GT87), cg0554 (GT4), cg1876 (GT4), cg1877 (GT4), Cg2400 (GT4) and egp_3191 (GT2).

In preferred embodiments, the Corynebacterium is Corynebacterium glutamicum. In embodiments, the Corynebacterium is suitable to produce fucosyllactose.

In embodiments, the genetically modified Corynebacterium of the invention exports fucosyllactose.

In embodiments of the invention, the genetically modified Corynebacterium is engineered to export fucosyllactose into the medium when grown at appropriate culture conditions. It is understood that export of FL relates to any kind of process, active or passive, that leads to FL being present outside the bacteria in the culture medium.

In embodiments, the Corynebacterium has been modified to express a permease for lactose import, such as lactose permease (LacY), GDP-D-mannose-4,6-dehydratase (GMD), GDP-L-fucose synthase (WcaG) and fucosyltransferase (FucT) from exogenous nucleic acid sequences.

In embodiments, the genetically modified Corynebacterium additionally comprise exogenous nucleic acids for expression of phosphomannomutase (ManB) and GTP-mannose-1-phosphate guanylyltransferase (ManC).

In embodiments, one or more of the exogenous nucleic acid sequences of the genetically modified bacteria are present in the bacterium as a plasmid. In embodiments, all of the exogenous nucleic acid sequences of the genetically modified bacteria are present in the bacterium as a plasmid.

In embodiments, one or more of the exogenous nucleic acid sequences of the genetically modified bacteria are chromosomally integrated. In embodiments, all the exogenous nucleic acid sequences of the genetically modified bacteria are chromosomally integrated.

In embodiments, the exogenous nucleic acid sequences encoding LacY, GMD, WcaG and FucT are chromosomally integrated. In embodiments, ManB ManC are chromosomally integrated.

All individual features disclosed and described above in the context of the genetically modified Corynebacterium of the invention with chromosomally integrated transgenes required for production of fucosyllactose are herewith also disclosed in the context of the genetically modified Corynebacterium of the invention for production of biomolecules, which is characterized in that the Corynebacterium is defective for functional expression of one or more glycosyltransferases involved in corynebacterial cell wall biosynthesis. The same holds true for a method of producing biomolecules using the bacteria of the present invention.

### BRIEF DESCRIPTION OF THE DRAWINGS

The invention is further described by the following figures. These are not intended to limit the scope of the invention but represent preferred embodiments of aspects of the invention provided for greater illustration of the invention described herein.

FIG. 1: Metabolic pathway representing the biosynthesis of 2-FucosylLactose engineered in C. glutamicum. A. C. glutamicum has no lactose transport capabilities, hence genes for Lactose transporters in addition to pathway enzymes are overexpressed under regulated and/or unregulated promotors to enhance the production of 2FL from glucose. (Ec=E. coli; Bf=Bacteroides fragilis; Ldb=Lactobacillus delbrueckii; Hp=Helicobacter pylori).

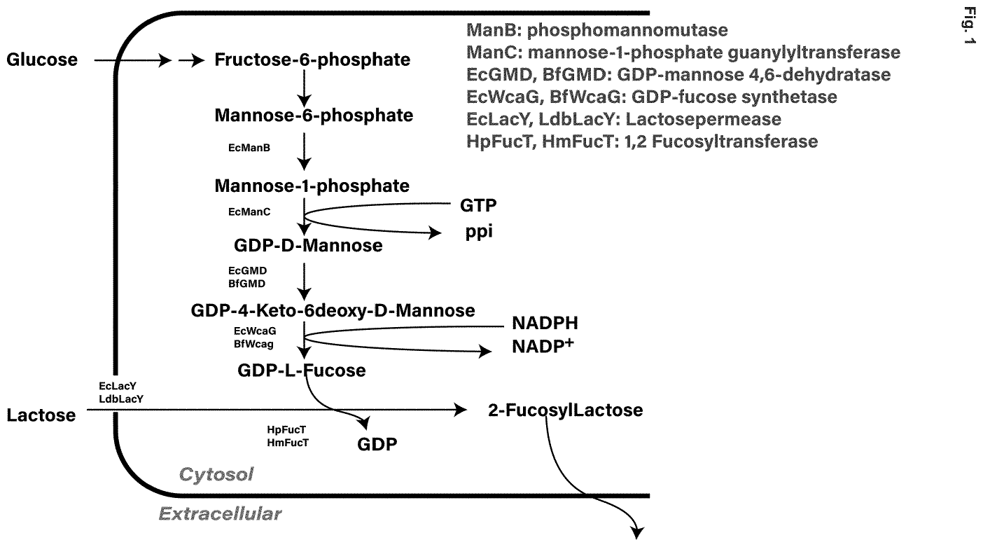

FIG. 2: Schematic representation of gene cassettes of exogenous nucleic acid sequences of the invention for chromosomal integration in the genetically modified corynebacteria of the invention.

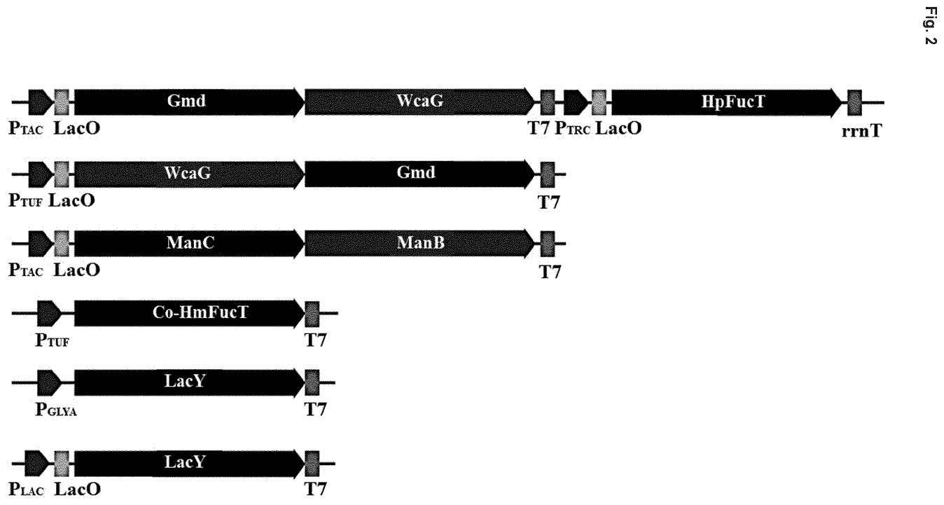

FIG. 3: Rational engineering of 2′-Fucosyllactose (2′-FL) production pathway in C. glutamicum. 2FL produced into culture supernatant by different C glutamicum strains after 72 h of shake flask cultivation in defined CGXII medium with 4% (w/v) glucose. All data were from biological triplicates, and error bars represent standard deviation. A. Comparison of different genetically engineered strains of the invention. B. Effect of deletion of different glycosyltransferases (MB13, MB14, MB15, MB17, MB20) compared to strain MB07. C. Intra and extracellular (into media) accumulation of 2′-FL in MB04 strain at the end of fedbatch fermentation in defined CGXII medium with 4% (w/v) glucose and 4 g/L lactose. Feeding medium contained 400 g/L glucose and 100 g/L (NH₄)₂SO₄, and the feed frequency was variably adjusted automatically depending on the DO signal. The feeding started when DO exceeds 30% and stops when DO felt again under the set-point. Samples collected were analyzed for 2FL production by HPLC.

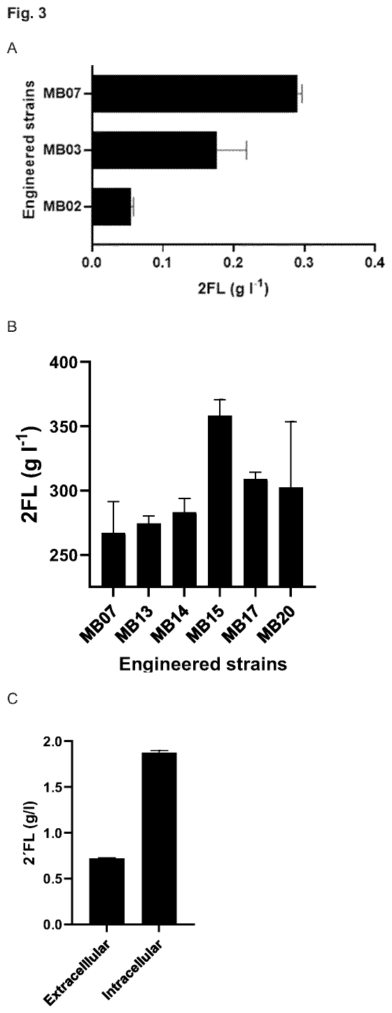

FIG. 4: Fedbatch fermentation. Fed-batch culture was performed with MB15 in a 1-L fermenter (Multifors, Infors) with 0.4 L working volume. The seed cultures were prepared in CGXII medium in shake flasks as described earlier; the fermenter was inoculated to an OD₆₀₀ of 1 in CGXII medium with 4% w/v glucose. The agitation and aeration were set at 800 rpm and 1 vvm, respectively. The pH was maintained at 7.0 and temperature at 30° C. throughout the fermentation. Dissolved oxygen (DO) was measured by an electrode, 100% was set by oxygen-saturated distilled water. 1 mM IPTG was added at t=16 h and 4 g/L lactose was added at t=25 h. Feeding medium contained 400 g/L glucose and 100 g/L (NH₄)₂SO₄, and the feed frequency was variably adjusted automatically depending on the DO signal. The feeding started when DO exceeds 30% and stops when DO felt again under the set-point. Samples collected were analyzed for 2FL production by HPLC.

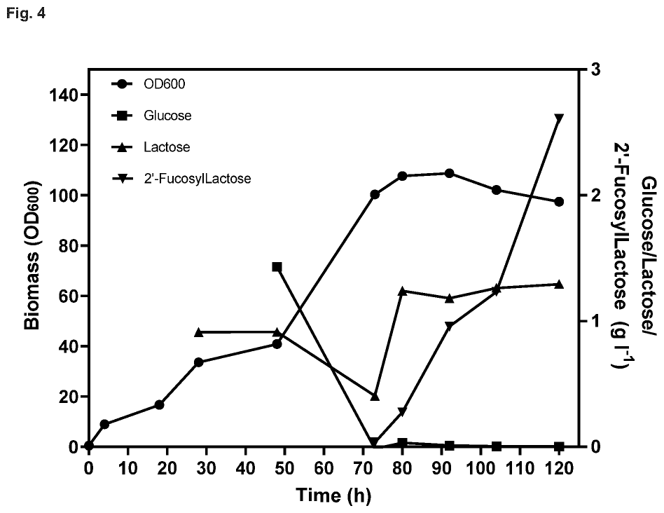

FIG. 5: Determination of extracellularly produced 2′-fucosyllactose (2-FL). 2-amino benzamide labeled 2′-fucosyllactose (2-FL) from media after Fed-batch fermentation of the engineered Corynebacterium glutamicum strain MB07 analyzed by HILIC-HPLC. HILIC-HPLC was performed using a TSK-Gel Amide-80 4.6×250 mm column with a linear gradient of solvent A (100% ACN) and solvent B (0.1% phosphoric acid) on an Agilent 1200 equipped with fluorescence detector. Fluorescence was measured at 420 nm with excitation at 320 nm (band width 16 nm).

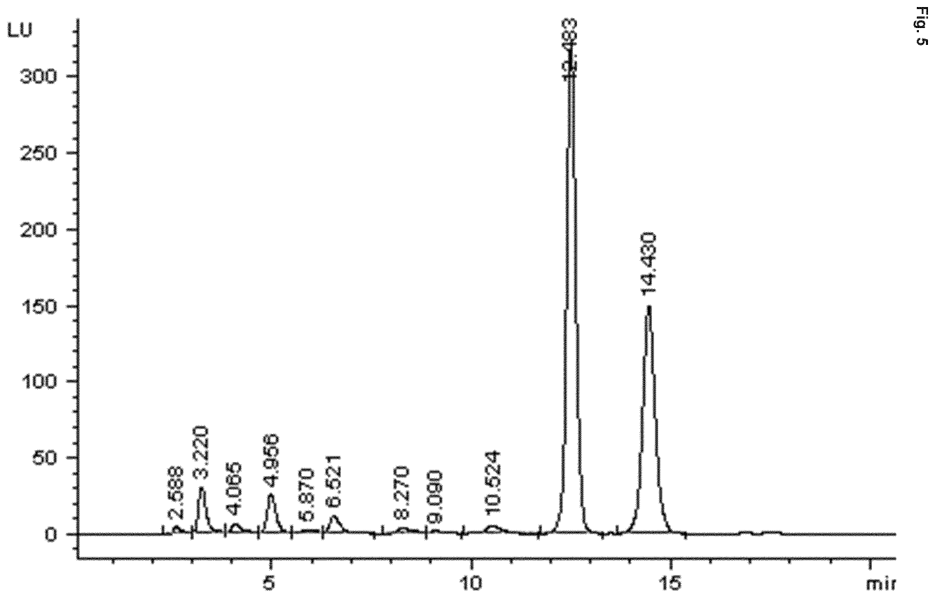

FIG. 6: The Figure shows the results of the Example below: Identification of positive candidates from experiments assessing the integration of lactose permease and beta-galactosidase reporter cassette in C. glutamicum by blue/white screening on X-Gal plates. Shown is a X-Gal plate with colonies of MB001 that were transformed by electroporation. Recombinants selected on Kanamycin plates grown overnight in BHI media, are diluted and plated on X-Gal LB plates with 10% sucrose. From left to right: Δcg3151::Lac_ZY, Δcg2854::Lac_ZY, Δcg0226::Lac_ZY (top) and Δcg2246::Lac_ZY (bottom), and Δcg2354::Lac_ZY. Qualitatively the strength of expression in each strain can be assessed by the intensity of the blue color of a colony.

FIG. 7: The Figure shows the results of the Example below. The graph depicts the beta-galactosidase activity (in Miller Units, Y-axis) of analyzed strains with the gene insertion at specified chromosomal sites (X-axis) of the integration of lactose permease and beta-galactosidase reporter cassette in C. glutamicum.

FIG. 8: The Figure shows the fed-batch culture of a production strain comprising the deletion of two glycosyltransferases (MB195). Fed-batch culture was performed with MB195 in a 2-L fermenter (Biostream, Netherlands) with 1 L working volume. The seed cultures were prepared in CGXII medium in shake flasks as described earlier; the fermenter was inoculated to an OD₆₀₀ of 1 in CGXII medium with 4% w/v glucose. The agitation and aeration were set at 800 rpm and 1 vvm, respectively. The pH was maintained at 7.0 and temperature at 30° C. throughout the fermentation. Dissolved oxygen (DO) was measured by an electrode, 100% was set by oxygen-saturated distilled water. 1 mM IPTG was added when biomass reached OD of 8 and 4 g/L lactose was added after two hours of induction. Feeding medium contained 400 g/L glucose and 100 g/L (NH₄)₂SO₄, and the feed frequency was adjusted to maintain glucose concentration at 5 g/. DO is set at 30% with stepwise increase of stirrer speed and aeration. Samples collected were analyzed for 2FL production by HPLC.

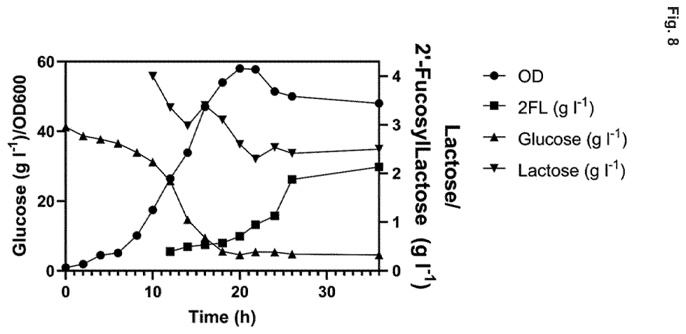

FIG. 9: The Figure depicts the results of the Example below, showing 2FL-yields (Y-axis) of deletion (engineered) strains (X-axis) with different glycosyltransferases in MB07. 2FL produced into culture supernatant by different C glutamicum strains after 72 h of shake flask cultivation in defined CGXII medium with 4% (w/v) glucose. All data were from biological triplicates, and error bars represent standard deviation.

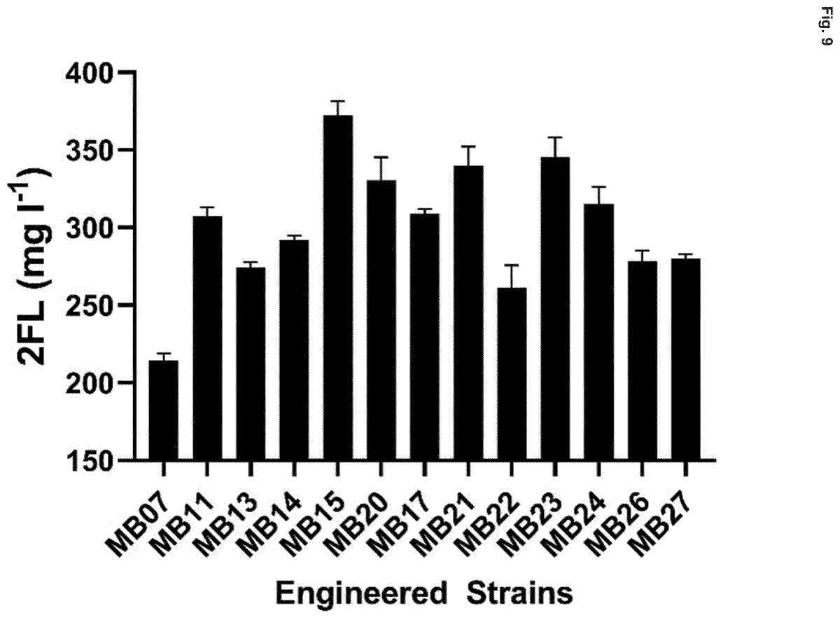

FIG. 10: The Figure depicts the results of the Example below. 2FL-yields (Y-axis) of double-deletion (GMO) strains (X-axis) with intracellular and extracellular levels of 2FL in comparison with MB07. The graph shows 2FL measurements in extracellular (white bars) and intracellular (black bars) media. 2′FL are the concentrations inside a cell, determined from lysed cell pellets per volume of samples cells of double GT deletion strains MB195 and MB143 compared to MB07. The data represents the mean (±standard deviation) of replicates of shake flask cultures. Black bars indicate intracellular and white bars indicate extracellular concentration.

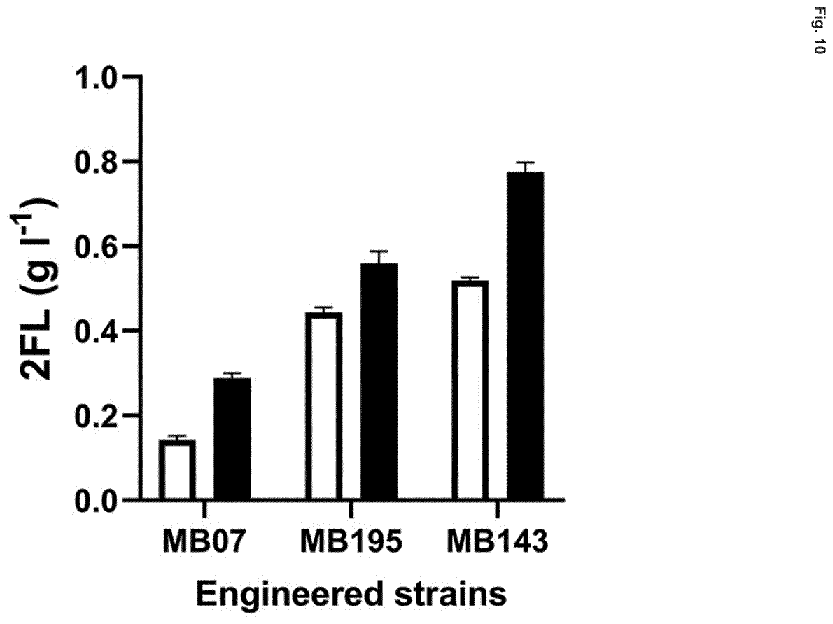

FIG. 11: The Figure shows the reduced foaming of a fed-batch fermentation with strain MB11 according to the invention.

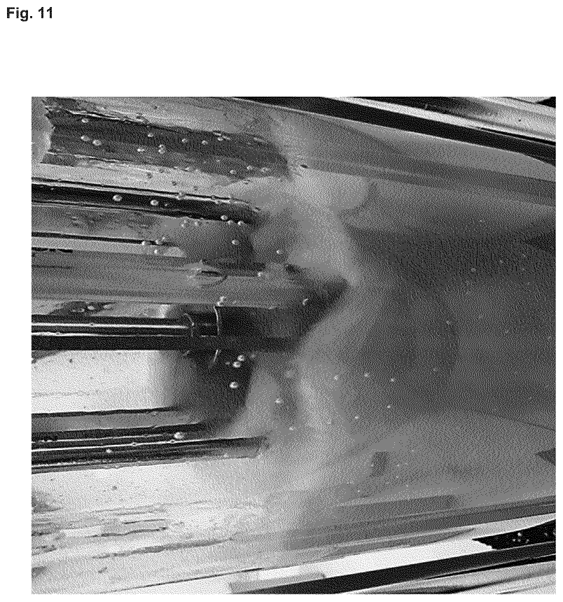

### DETAILED DESCRIPTION OF THE INVENTION

All cited documents of the patent and non-patent literature are hereby incorporated by reference in their entirety.

In one aspect, the present invention relates to genetically modified (or engineered) Corynebacterium for production of fucosyllactose, wherein the Corynebacterium has been modified to express a permease for lactose import, GDP-D-mannose-4,6-dehydratase (GMD), GDP-L-fucose synthase (WcaG) and fucosyltransferase (FucT) from exogenous nucleic acid sequences, characterized in that the exogenous nucleic acid sequences encoding a permease for lactose import, GMD, WcaG and FucT are chromosomally integrated.

Corynebacterium is a genus of bacteria that are Gram-positive and aerobic. They are bacilli (rod-shaped), and in some phases of life they are club-shaped, which inspired the genus name (coryneform means “club-shaped”). They are widely distributed in nature in the microbiota of animals (including the human microbiota) and are mostly innocuous, most commonly existing in commensal relationships with their hosts. Corynebacteria are useful in industrial settings in particular Corynebacterium glutamicum.

16 conserved signature proteins, which are uniquely found in Corynebacterium species, have been identified. Three of the conserved signature proteins have homologs found in the genus Dietzia, which is believed to be the closest related genus to Corynebacterium. In phylogenetic trees based on concatenated protein sequences or 16S rRNA, the genus Corynebacterium forms a distinct clade, within which is a distinct subclade, cluster I. The cluster is made up of the species C. diptheriae, C. pseudotuberculosis, C. ulcerans, C. aurimucosum, C. glutamicum, and C. efficiens. This cluster is distinguished by several conserved signature indels, such as a two-amino-acid insertion in LepA and a seven- or eight-amino-acid insertions in RpoC. Also, 21 conserved signature proteins are found only in members of cluster I. Another cluster has been proposed, consisting of C. jeikeium and C. urealyticum, which is supported by the presence of 19 distinct conserved signature proteins which are unique to these two species. Corynebateria have a high G+C content ranging from 46-74 mol %.

Corynebateria are gram-positive, catalase-positive, non-spore-forming, non-motile, rod-shaped bacteria that are straight or slightly curved. Metachromatic granules are usually present representing stored phosphate regions. Their size falls between 2 and 6 μm in length and 0.5 μm in diameter. The bacteria group together in a characteristic way, which has been described as the form of a “V”, “palisades”, or “Chinese characters”. They may also appear elliptical. They are aerobic or facultatively anaerobic, chemoorganotrophs. They are pleomorphic through their lifecycles, they occur in various lengths, and they frequently have thickenings at either end, depending on the surrounding conditions.

The Corynebateria cell wall is distinctive, with a predominance of mesodiaminopimelic acid in the murein wall and many repetitions of arabinogalactan, as well as corynemycolic acid (a mycolic acid with 22 to 26 carbon atoms), bound by disaccharide bonds called L-Rhap-(1→4)-D-GlcNAc-phosphate. These form a complex commonly seen in Corynebacterium species: the mycolyl-AG-peptidoglican (mAGP).

Corynebacteria grow slowly, even on enriched media. In terms of nutritional requirements, all need biotin to grow. Some strains also need thiamine and PABA. Some of the Corynebacterium species with sequenced genomes have between 2.5 and 3.0 million base pairs. The bacteria grow in Loeffler's medium, blood agar, and trypticase soy agar (TSA). They form small, grayish colonies with a granular appearance, mostly translucent, but with opaque centers, convex, with continuous borders. The color tends to be yellowish-white in Loeffler's medium. In TSA, they can form grey colonies with black centers and dentated borders that look similar to flowers (C. gravis), or continuous borders (C. mitis), or a mix between the two forms (C. intermedium).

Nonpathogenic species of Corynebacterium are used for very important industrial applications, such as the production of amino acids, nucleotides, and other nutritional factors; bioconversion of steroids; degradation of hydrocarbons; cheese aging; and production of enzymes. Some species produce metabolites like antibiotics: bacteriocins of the corynecin-linocin type, antitumor agents, among others. One of the most studied species is C. glutamicum, whose name refers to its capacity to produce glutamic acid in aerobic conditions. Species of Corynebacterium and in particular C. glutamicum have been used in the mass production of various amino acids including glutamic acid, a food additive that is made at a rate of 1.5 million tons/year. The metabolic pathways of Corynebacterium have been further manipulated to produce lysine and threonine. L-Lysine production is specific to C. glutamicum in which core metabolic enzymes are manipulated through genetic engineering to drive metabolic flux towards the production of NADPH from the pentose phosphate pathway, and L-4-aspartyl phosphate, the commitment step to the synthesis of L-lysine, lysC, dapA, dapC, and dapF. These enzymes are up regulated in industry through genetic engineering to ensure adequate amounts of lysine precursors are produced to increase metabolic flux. Unwanted side reactions such as threonine and asparagine production can occur if a buildup of intermediates occurs, so scientists have developed mutant strains of C. glutamicum through PCR engineering and chemical knockouts to ensure production of side-reaction enzymes are limited. Many genetic manipulations conducted in industry are by traditional cross-over methods or inhibition of transcriptional activators.

Unlike gram-negative bacteria, the gram-positive Corynebacterium species lack lipopolysaccharides that function as antigenic endotoxins in humans, which is highly advantageous to produce biomolecules intended for consumption by humans.

Corynebacterium species include nonlipophilic Corynebacteria that may be classified as fermentative and nonfermentative. Fermentative corynebacteria include Corynebacterium diphtheriae group, Corynebacterium xerosis and Corynebacterium striatum, Corynebacterium minutissimum, Corynebacterium amycolatum, Corynebacterium glucuronolyticum, Corynebacterium argentoratense, Corynebacterium matruchotii, Corynebacterium glutamicum, Corynebacterium sp. Nonfermentative corynebacteria include Corynebacterium afermentans subsp. Afermentans, Corynebacterium auris, Corynebacterium pseudodiphtheriticum, Corynebacterium propinquum. Lipophilic Corynebacteria include Corynebacterium uropygiale, Corynebacterium jeikeium, Corynebacterium urealyticum, Corynebacterium afermentans subsp. Lipophilum, Corynebacterium accolens, Corynebacterium macginleyi, CDC coryneform groups F-1 and G, Corynebacterium bovis. This list of Corynebacteria is non limiting for the scope of the invention.

In embodiments, the Corynebacterium of the invention is a non-pathogenic species or strain. In embodiments, the Corynebacterium is a strain that is considered a GRAS host. In preferred embodiments, the Corynebacterium is C. glutamicum.

Corynebacterium glutamicum (previously known as Micrococcus glutamicus) is a Gram-positive, rod-shaped bacterium that is used industrially for large-scale production of amino acids. While originally identified in a screen for organisms secreting L-glutamate, mutants of C. glutamicum have also been identified that produce various other amino acids. Due to its industrial importance, several clones of C. glutamicum have been sequenced. Furthermore, small RNA data was obtained by RNA-Seq in C. glutamicum ATCC 13032. Corynebacterium glutamicum ATCC 14067 was previously known as Brevibacterium flavum.

Furthermore, with the sequencing of the whole genome of C. glutamicum, new methods such as proteomics, metabolomics and transcriptome analyses were established that have led to a variety of approaches in metabolic engineering to rationally construct/improve production strains.

Stable maintenance of expression plasmids is unfavorable in industrial production strains because antibiotics must be supplied to the cultivation media. Consequently, heterologous genes are integrated, or native genes are deleted in the genome of the respective organism. The insertion, deletion or substitution of genes in C. glutamicum is usually performed by homologous recombination using non-replicative integration vectors and genome modification can be achieved via two rounds of positive selection, as is known to the skilled person. One example method of this kind of genetic modification uses the non-replicative plasmid pK19mobsacB, which can preferably be used to generate the corynebacterial of the present invention. Furthermore, targeted genome modification in C. glutamicum can also be performed via conjugation by using E. coli vectors carrying manipulated C. glutamicum DNA fragments.

The present invention relates to genetically modified (or genetically engineered) Corynebacteria. Therein, the term “genetically modified” describes bacteria whose genetic material has been modified in comparison to a naturally occurring wild type strain, for example by deleting or removing genetic elements of the wild type strain and/or by inserting additional genetic material, in particular DNA sequences, for example in form of non-integrating DNA plasmids or as DNA sequences that chromosomally integrate into the bacterial genome. Genetic modifications also include deletions of chromosomal sequences or other genetic elements of the bacteria. Possible techniques of genetically modifying Corynebacterium and in particular C. glutamicum are described herein. As used herein, transgenic bacteria are genetically modified bacteria. A transgenic bacterium is a bacterium that comprises an exogenous nucleic acid molecule for expression of a protein, which is preferably from another organism or species, for example.

In one aspect, the Corynebacterium of the invention is to produce fucosyllactose. In preferred embodiments, the fucosyllactose comprises 2-Fucosyllactose (2-FL or 2FL). In further embodiments, fucosyllactose comprises 3-Fucosyllactose (3-FL or 3FL).

2-FL (IUPAC name (2R,3R,4R,5R)-4-[(2S,3R,4S,5R,6R)-4,5-dihydroxy-6-(hydroxymethyl)-3-[(2S,3S,4R,5S,6S)-3,4,5-trihydroxy-6-methyloxan-2-yl]oxyoxan-2-yl]oxy-2,3,5,6-tetrahydroxyhexanal) is an oligosaccharide, more precisely, fucosylated, neutral trisaccharide composed of L-fucose, D-galactose, and D-glucose units. It is the most prevalent human milk oligosaccharide (HMO) naturally present in human breast milk, making up about 30% of all of HMOs.

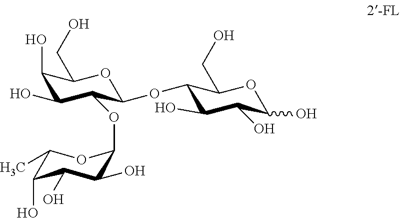

As with other oligosaccharides, a widely regarded characteristic of 2′-fucosyllactose is its ability to protect against infectious diseases namely in preventing epithelial level adhesions of toxins and pathogens. The 2FL stimulates the growth of certain Bifidobacteria and receptor analogons which lends to toxic and pathogenic protection, all this being most prevalent in infants. Among the pathogens that 2FL is known to protect against are Campylobacter jejuni, Salmonella enterica serotype Typhimurium, Helicobacter pylori, among others.

3-FL (3-Fucosyllactose) is one of the most abundant fucosylated HMOs, and particularly dominant in the milk of non-secretor mothers. 3-FL occurs in human milk in a concentration of 0.72 (+0.7) g/L. While the concentration of 2-FL decrease over the lactation time, the amount of 3-FL increases in later lactation stages. The specific functional benefits of 3-FL include reducing the risk of infection by inhibiting the adhesion of pathogenic bacteria, e.g. Pseudomonas aeruginosa or enteropathogenic E. coli or viruses e.g. Norovirus. It also shows general ability to reduce colonization of non-beneficial bacteria, e.g. Enterococcus faecium, and in turn positively supporting gut health by selectively stimulating beneficial bifidobacterial. Further studies on 3-FL suggest positive effects in regulating intestinal motility.

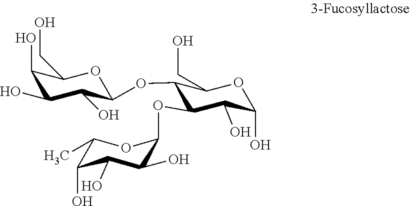

For engineering a fucosyllactose biosynthesis pathway in the Corynebacterium, the Corynebacterium of the invention can comprise an exogenous nucleic acid sequence encoding and enabling expression of a fucosyltransferase (FucT).

Fucosyltransferases belong to Leloir glycosyltransferases, as these enzymes use a nucleotide-activated sugar (GDP-fucose (guanosine diphosphate-fucose) as glycosyl donor for their reaction. In the carbohydrate active enzymes database (CAZy, http://www.cazy.org), which classifies glycosyltransferases based on amino acid sequence homology, fucosyltransferases are classified under glycosyltransferase families GT-10, GT-11, GT-23, GT-37, GT-65, GT-68 and GT-74. The FucTs relevant for HMO synthesis belong to GT-10 (α1,3-, and/or α1,4-FucTs) or GT-11 (α1,2-FucTs) and GT 74 (α1,2-FucT). Fucosyltransferases (FucTs, EC 2.4.1.x) are categorized into α1,2-, α1,3/4-, and α1,6-selective enzyme groups, depending on the site selectivity of the enzyme in the reaction with the acceptor carbohydrates. The acceptor substrate can be another sugar such as the transfer of a fucose to a core GlcNAc (N-acetylglucosamine) sugar as in the case of N-linked glycosylation, or to a protein, as in the case of O-linked glycosylation produced by O-fucosyltransferase. There are various fucosyltransferases in mammals, the vast majority of which, are located in the Golgi apparatus. The O-fucosyltransferases have recently been shown to localize to the endoplasmic reticulum (ER). (Brockhausen, Inka. “Crossroads between Bacterial and Mammalian Glycosyltransferases.” Frontiers in Immunology, vol. 5, 2014, pp. 492-492; Petschacher, Barbara, and Bernd Nidetzky. “Biotechnological Production of Fucosylated Human Milk Oligosaccharides: Prokaryotic Fucosyltransferases and Their Use in Biocatalytic Cascades or Whole Cell Conversion Systems.” Journal of Biotechnology, vol. 235, 2016, pp. 61-83.)

As used herein, the term “fucosyltransferase” comprises in particular “alpha-1,2-fucosyltransferase” (FucT2) and “alpha-1,3-fucosyltransferase” (FucT3). A fucosyltransferase or a nucleic acid sequence/polynucleotide encoding an fucosyltransferase refer to a glycosyltransferase that catalyzes the transfer of fucose from a donor substrate, for example, GDP-fucose, to an acceptor molecule, preferably in an alpha-1,2-linkage or alpha-1,3-linkage, respectively. The acceptor molecule can be a carbohydrate, an oligosaccharide, a protein or glycoprotein, or a lipid or glycolipid, and can be, e.g., N-acetylglucosamine, N-acetyllactosamine, galactose, fucose, sialic acid, glucose, lactose or any combination thereof. In the context of the present invention, the acceptor molecule is preferably lactose.

Preferred fucosyltransferases for use in the context of the invention comprise FucT from Helicobacter pylori (NCBI-Protein ID AAD29863.1, UniProt Q9X435) and FucT from Helicobacter mustelae (NCBI-Protein ID CBG40460.1, UniProt D3UIY5) and alpha-1,2-FucT WbsJ from E. coli 0128: B12. However, Genera of organisms with know FucT activities (both α-1,2- and α-1,3 FucTs) suited for use in the context of the present invention comprise Helicobacter, Escherichia, Bacteroides, Anopheles, Apis, Caenorhabditis, Cricetulus, Danio, Rattus, Gallus, Medicago, Sus, Oryza, Physcomitrium, Gallus, Mus, Bos, Schistosoma, Canis, Vigna, Zea, Eulemur Gorilla, Hylobates, Macaca, Oryctolagus, Pan, Pongo, Thermosynechococcus, Dictyostelium, Arabidopsis, Drosophila, and Homo. A complete list of suitable FucT enzymes known so far is disclosed on www.cazy.org (Carbohydrate-Active enZYmes Database describing families of structurally-related catalytic and carbohydrate-binding modules (or functional domains) of enzymes that degrade, modify, or create glycosidic bonds), specifically http://www.cazy.org/GT10_characterized.html (for GlycosylTransferase Family 10 comprising various FucT3 from many different organisms) and http://www.cazy.org/GT11_characterized.html (for GlycosylTransferase Family 11 comprising various FucT2 from many different organisms).

Few FucTs from prokaryotic and eukaryotes have been characterized. Examples include α-FucTs from H. pylori, H. Mustulae, E. coli 086: K62: H2, E. coli 086: B7, E. coli O128: B12, E. coli O127: K63 (B8), E. coli 0126 and Thermosynechococcus elongatus, and α-1,3/4-FucTs from H. pylori, H. hepaticus and B. fragilis.

In embodiments, the one or more FucT expressed by the Corynebacterium of the invention is selected from the group comprising α-FucTs from H. pylori, H. Mustulae, E. coli O86: K62: H2, E. coli 086: B7, E. coli O128: B12, E. coli O127:K63 (B8), E. coli 0126 and Thermosynechococcus elongatus. α-1,3/4-FucTs from H. pylori, H. hepaticus and B. fragilis.

In preferred embodiments, one or more FucT expressed by the Corynebacterium of the invention is selected from the group comprising α-FucTs from H. pylori, H. Mustulae, E. coli O126 and Thermosynechococcus elongatus and α-1,3/4-FucTs from H. pylori, H. hepaticus and B. fragilis.

In preferred embodiments, one or more FucT expressed by the Corynebacterium of the invention is selected from the group comprising α-FucTs from H. pylori, H. Mustulae, E. coli O126 and Thermosynechococcus elongatus.

In preferred embodiments, one or more FucT expressed by the Corynebacterium of the invention is selected from the group comprising α-1,3/4-FucTs from H. pylori, H. hepaticus and B. fragilis.

Furthermore, for engineering a fucosyllactose biosynthesis pathway in the Corynebacterium, the Corynebacterium comprise in embodiments an exogenous nucleic acid sequences encoding and enabling expression of GDP-D-mannose-4,6-dehydratase (GMD).

A GDP-mannose 4,6-dehydratase (GMD) is an enzyme that catalyzes the chemical reaction GDP-mannose=GDP-4-dehydro-6-deoxy-D-mannose+H2O. Hence, this enzyme has one substrate, GDP-mannose, and two products, GDP-4-dehydro-6-deoxy-D-mannose and H2O. Known GMDs from different organisms that can be used in the context of the present invention are disclosed on https://enzyme.expasy.org/EC/4.2.1.47), for example.

GMD belongs to the family of lyases, specifically the hydro-lyases, which cleave carbon-oxygen bonds. The systematic name of this enzyme class is GDP-mannose 4,6-hydro-lyase (GDP-4-dehydro-6-deoxy-D-mannose-forming). Other names in common use include guanosine 5′-diphosphate-D-mannose oxidoreductase, guanosine diphosphomannose oxidoreductase, guanosine diphosphomannose 4,6-dehydratase, GDP-D-mannose dehydratase, GDP-D-mannose 4,6-dehydratase, Gmd, and GDP-mannose 4,6-hydro-lyase. This enzyme participates in fructose and mannose metabolism. It employs one cofactor, NAD+.

GMD is present in the GDP-mannose-dependent de novo pathway which provides GDP-Fucose. In the pathway the enzyme is in an intermediate step that converts GDP-Mannose to GDP-4-dehydro-6-deoxy-D-mannose which is then subsequently converted into GDP-Fucose. The product of this pathway is used by fucosyltransferases.

For engineering a fucosyllactose biosynthesis pathway in the Corynebacterium, the Corynebacterium comprise in embodiments an exogenous nucleic acid sequences encoding and enabling expression of GDP-L-fucose synthase (WcaG).

A GDP-L-fucose synthase (WcaG) is an enzyme that catalyzes the following chemical reaction:

GDP-4-dehydro-6-deoxy-D-mannose+NADPH+H+⇄GDP-L-fucose+NADP+

Thus, the three substrates of WcaG are GDP-4-dehydro-6-deoxy-D-mannose, NADPH, and H+, whereas its two products are GDP-L-fucose and NADP+. WcaG belongs to the family of oxidoreductases, specifically those acting on the CH—OH group of donors with NAD+ or NADP+ as acceptor. The systematic name of this enzyme class is GDP-L-fucose: NADP+4-oxidoreductase (3,5-epimerizing). This enzyme is also called GDP-4-keto-6-deoxy-D-mannose-3,5-epimerase-4-reductase. WcaG participates in the GDP-mannose-dependent de novo pathway which provides GDP-Fucose. Therein, WcaG converts GDP-4-dehydro-6-deoxy-D-mannose into GDP-Fucose and therefore acts downstream of GMD. Known GMDs and WcaGs from different organisms that can be used in the context of the present invention are disclosed on https://enzyme.expasy.org/EC/4.2.1.47 and https://enzyme.expasy.org/EC/1.1.1.271, respectively, for example.

Furthermore, in embodiments the Corynebacterium comprise a permease for lactose import. Such permeases comprise various lactose permeases, which are membrane proteins that are members of the major facilitator superfamily. Lactose permease can be classified as a symporter, which uses the proton gradient towards the cell to transport β-galactosides such as lactose in the same direction into the cell. The protein has twelve transmembrane alpha-helices and its molecular weight is about 45,000 Daltons. It exhibits an internal two-fold symmetry, relating the N-terminal six helices onto the C-terminal helices. It is encoded by the lacY gene in the lac operon of various bacteria, such as E. coli. The present invention is not limited to specific Lactose permeases, as long as lactose import into the Corynebacterium can be ensured by expression of the lactose permease from the exogenous nucleic acid sequence. A skilled person can identify suitable lactose permeases by using well established functional tests. Preferred lactose permeases include LacY from E. coli and LacS from Lactobacillus delbrueckii and Streptococcus thermophilus as disclosed in the examples.

In embodiments, the Corynebacterium comprise exogenous nucleic acid sequences encoding and enabling expression of phosphomannomutase (ManB).

A phosphomannomutase (ManB) is an enzyme that catalyzes the following chemical reaction:

alpha-D-mannose 1-phosphate⇄D-mannose 6-phosphate

Hence, this enzyme has one substrate, alpha-D-mannose 1-phosphate, and one product, D-mannose 6-phosphate. ManB belongs to the family of isomerases, specifically the phosphotransferases (phosphomutases), which transfer phosphate groups within a molecule. The systematic name of this enzyme class is alpha-D-mannose 1,6-phosphomutase. Other names in common use include mannose phosphomutase, phosphomannose mutase, and D-mannose 1,6-phosphomutase. ManB participates in fructose and mannose metabolism. It has 2 cofactors: D-glucose 1,6-bisphosphate, and D-Mannose 1,6-bisphosphate. Known ManB variants from different organisms that can be used in the context of the present invention are disclosed on https://enzyme.expasy.org/EC/5.4.2.8, for example.

In embodiments, the Corynebacterium comprise exogenous nucleic acid sequences encoding and enabling expression of GTP-mannose-1-phosphate guanylyltransferase (ManC).

GTP-mannose-1-phosphate guanylyltransferase (ManC) (or mannose-1-phosphate guanylyltransferase) is an enzyme that catalyzes the following chemical reaction:

GTP+alpha-D-mannose 1-phosphate⇄diphosphate+GDP-mannose

Thus, the two substrates of this enzyme are GTP and alpha-D-mannose 1-phosphate, whereas its two products are diphosphate and GDP-mannose. This enzyme belongs to the family of transferases, specifically those transferring phosphorus-containing nucleotide groups (nucleotidyltransferases). The systematic name of this enzyme class is GTP: alpha-D-mannose-1-phosphate guanylyltransferase. Other names in common use include GTP-mannose-1-phosphate guanylyltransferase, PIM-GMP (phosphomannose isomerase-guanosine 5′-diphospho-D-mannose, pyrophosphorylase), GDP-mannose pyrophosphorylase, guanosine 5′-diphospho-D-mannose pyrophosphorylase, guanosine diphosphomannose pyrophosphorylase, guanosine triphosphate-mannose 1-phosphate guanylyltransferase, and mannose 1-phosphate guanylyltransferase (guanosine triphosphate). This enzyme participates in fructose and mannose metabolism. Known ManC variants from different organisms that can be used in the context of the present invention are disclosed on https://enzyme.expasy.org/EC/2.7.7.13, for example.

Preferred nucleic acid sequences of the invention encoding preferred variants of the enzymes that can be exogenously expressed in embodiments of the bacteria of the invention are provided in Table 1.

TABLE 1
Preferred nucleic acid sequences encoding transgenes of the
corynebacterium of the invention.

| | |
|---|---|
| SEQ ID NO 1: ManB | ATGTCGAGTTTAACCTGCTTTAAAGCCTATGATATTCG |
| (corresponding to NCBI- | CGGGAAATTAGGCGAAGAACTGAATGAAGATATCGCC |
| ProteinID ADT75633, and | TGGCGTATTGGGCGTGCCTATGGCGAATTTCTCAAACC |
| UniProt EOJ145) | GAAAACCATTGTGTTAGGCGGTGACGTCCGCCTCACCA |
| | GCGAAACCTTAAAACTGGCGCTGGCGAGAGGTTTACA |
| | GGATGCGGGCGTCGATGTGCTGGATATCGGCATGTCCG |
| | GCACCGAAGAGATCTATTTCGCCACGTTCCATCTCGGC |
| | GTGGATGGCGGCATCGAAGTTACCGCCAGCCATAATCC |
| | GATGGATTATAACGGCATGAAGCTGGTTCGCGAGGGG |
| | GCTCGCCCGATCAGCGGAGATACCGGACTGCGCGACG |
| | TCCAGCGTCTGGCTGAAGCCAACGACTTTCCTCCCGTC |
| | GATGAAACCAAACGCGGTCGCTATCAGCAAATCAACC |
| | TGCGTGACGCTTACGTTGATCACCTGTTCGGTTATATCA |
| | ATGTCAAAAACCTCACGCCGCTCAAGCTGGTGATCAAC |
| | TCCGGGAACGGCGCAGCGGGTCCGGTGGTGGACGCTA |
| | TCGAAGCCCGCTTTAAAGCCCTCGGCGCACCTGTGGAA |
| | TTGATCAAAGTGCACAACACGCCGGACGGCAATTTCCC |
| | CAACGGTATTCCTAACCCGCTGCTGCCGGAATGTCGCG |
| | ACGACACCCGCAATGCGGTCATCAAACACGGCGCGGA |
| | TATGGGCATTGCCTTTGATGGCGATTTTGACCGCTGTTT |
| | CCTGTTTGACGAAAAAGGGCAGTTTATCGAGGGCTACT |
| | ACATTGTCGGCCTGCTGGCAGAAGCGTTCCTCGAAAAA |
| | AATCCCGGCGCGAAGATCATCCACGATCCACGTCTCTC |
| | CTGGAATACCGTTGATGTGGTGACCGCCGCGGGCGGCA |
| | CACCGGTGATGTCGAAAACCGGACACGCCTTTATTAAA |
| | GAACGTATGCGCAAGGAAGACGCCATCTACGGTGGCG |
| | AAATGAGCGCCCACCACTATTTCCGTGATTTCGCTTAC |
| | TGCGACAGCGGCATGATCCCGTGGCTGCTGGTCGCCGA |
| | ACTGGTGTGTCTGAAAGGAAAAACGCTGGGCGAACTG |
| | GTGCGCGACCGGATGGCAGCGTTTCCGGCAAGCGGTG |
| | AGATCAACAGCAAACTGGCGCAACCCGTTGAGGCGAT |
| | TAATCGCGTCGAACAGCATTTTAGCCGCGAGGCGCTGG |
| | CGGTGGATCGCACCGATGGCATCAGCATGACCTTTGCC |
| | GACTGGCGCTTTAACCTGCGCTCCTCCAACACCGAACC |
| | GGTGGTGCGGTTGAATGTGGAATCGCGCGGTGATGTGC |
| | CGCTGATGGAAGAAAAGACAAAACTTATCCTTGCGTTA |
| | TTGAACAAGTAA |
| SEQ ID NO 2: ManC | ATGAACAATAATAAAATTATTACACCTATCATTATGGC |
| (corresponding to NCBI- | AGGTGGTTCAGGCAGTCGGTTGTGGCGACTATCAAGAA |
| ProteinID ADT75634, and | TTCTCTATCCGAAACAATTTCTTAGCCTAAACGGTAGT |
| UniProt E8PZ98) | CATACCATGCTTCAAACAACGGCTAATCGTCTGGATGG |
| | TTTGGATTGTACCAACCCTTATGTCATTTGTAATGAACA |
| | ACACCGCTTTATAGTTGCTGAACAGCTTAGAAAAATCG |
| | ATAGATTGACTTCAAAGAATATCATCCTTGAGCCTGTT |
| | GGGCGTAACACTGCCCCTGCAATTGCATTAGCGGCGTT |
| | GCTGATGTCTAAGTCTGATAAAAGTGCAGATGATCTTA |
| | TGCTCGTACTGGCTGCAGATCACGTTATATTAGATGAA |
| | GAAAAATTTTGTAACGCTGTTAGATCGGCAATTCCATA |
| | CGCTGCTGATGGGAAATTGGTAACATTTGGTATAATTC |
| | CAGACAAAGCAGAAACTGGTTATGGTTATATACATCGA |
| | GGACAATATATTAATCAGGAAGATTCGGATGCATTTAT |
| | AGTGTCATCATTTGTTGAAAAGCCAAATCATGAGACAG |
| | CCACTAAATATCTTGCTTCCGGTGAGTATTATTGGAAT |
| | AGCGGTATGTTTTTGTTTAGTGCAAATCGTTATATAGA |
| | GGAACTTAAACAATTTCGGCCTGATATTTTATCCGCTT |
| | GTGAAAAAGCAATTGCTTCAGCGAACTTTGACCTTGAT |
| | TTTGTGCGTTTAGATGAAAGTTCTTTCTCTAAGTGTCCT |
| | GAAGAGTCAATTGATTACGCTGTAATGGAAAAAACAA |
| | AAGACGCAATTGTTATTCCAATGGATGCTGGCTGGAGT |
| | GATGTCGGTTCATGGTCTTCTCTTTGGGAAATTAATGAT |
| | AAAGACTCAGACGGCAACGTAATAGTTGGGGATATTTT |
| | CTCTCATGAAACAAAGAATTCTTTCATATATGCCGAAT |
| | CGGGAATTGTTGCTACAGTGGGAGTGGAAAATTTAGTT |
| | GTTGTCCAAACAAAGGATGCCGTTCTTGTCTCAGAGAG |
| | AAATAAAGTTCAGGATGTAAAGAAAATAGTCGAACAA |
| | ATTAAAAATTCAGGTCGTAGCGAGCATTATGTTCATCG |
| | CGAAGTATATCGTCCTTGGGGTAAATATGATTCCATTG |
| | ACACAGGGGAGCGTTATCAGGTCAAACGTATAACAGT |
| | AAATCCTGGTGAAGGACTTTCTTTACAAATGCACCATC |
| | ATAGGGCAGAACATTGGATCATAGTTTCTGGAACTGCA |
| | AAGGTGACTATAGGTTCTGAAACTAAGATTCTTAGCGA |
| | AAATGAATCTGTTTACATACCTCTTGGTGTAATACACT |
| | GCTTGGAAAATCCAGGGAAAATTCCTCTTGATTTAATT |
| | GAAGTTCGTTCTGGATCTTATTTAGAAGAAGACGATGT |
| | TATCCGTTTTCAGGACCGATATGGTCGCAGCTAA |
| SEQ ID NO 3: GMD from | ATGTCAAAAGTCGCTCTCATCACCGGTGTAACCGGACA |
| E. coli (GMD_EC) | AGACGGTTCTTACCTGGCAGAGTTTCTGCTGGAAAAAG |
| (corresponding to NCBI- | GTTACGAGGTGCATGGTATTAAGCGTCGTGCATCGTCC |
| ProteinID ADT75653, and | TTCAACACCGAGCGCGTGGATCACATTTATCAGGATCC |
| UniProt E0J125) | GCACACCTGCAACCCGAAATTCCATCTGCATTATGGCG |
| | ACCTGAGTGATACCTCCAACCTGACGCGCATTTTGCGT |
| | GAAGTGCAGCCGGATGAAGTGTACAACCTGGGCGCAA |
| | TGAGTCACGTTGCGGTTTCTTTTGAGTCACCGGAATAT |
| | ACCGCAGACGTCGATGCGATGGGTACGCTGCGCCTGCT |
| | GGAGGCGATCCGCTTCCTCGGTCTGGAAAAGAAAACC |
| | CGTTTCTATCAGGCTTCCACCTCTGAACTGTACGGTCTG |
| | GTGCAGGAAATTCCGCAGAAAGAAACCACGCCGTTCT |
| | ACCCGCGATCTCCGTATGCGGTCGCCAAACTGTATGCC |
| | TACTGGATCACCGTTAACTACCGCGAATCCTACGGCAT |
| | GTACGCCTGTAACGGTATTCTCTTCAACCATGAATCCC |
| | CGCGCCGCGGCGAAACCTTCGTTACCCGCAAAATCACC |
| | CGCGCAATCGCCAACATCGCCCAGGGGCTGGAGTCGT |
| | GCCTGTACCTCGGCAATATGGATTCCCTGCGTGACTGG |
| | GGCCACGCCAAAGACTACGTAAAAATGCAGTGGATGA |
| | TGCTGCAACAGGAACAGCCGGAAGATTTCGTTATCGCG |
| | ACCGGCGTTCAGTACTCCGTGCGTCAGTTCGTGGAAAT |
| | GGCAGCGGCACAGCTGGGCATCAAACTGCGCTTTGAA |
| | GGCACGGGTGTTGAAGAGAAGGGCATTGTGGTTTCCGT |
| | CACCGGGCATGACGCGCCGGGCGTTAAACCGGGTGAT |
| | GTGATTATCGCCGTTGATCCGCGTTACTTCCGTCCGGCT |
| | GAAGTTGAAACGCTGCTCGGCGACCCGACCAAAGCGC |
| | ACGAAAAACTGGGCTGGAAACCGGAAATCACCCTCAG |
| | AGAGATGGTGTCTGAAATGGTGGCTAATGACCTCGAA |
| | GCGGCGAAAAAACACTCTCTGCTGAAATCTCACGGCTA |
| | CGACGTGGCGATCGCGCTGGAGTCATAA |
| SEQ ID NO 4: WcaG | ATGAGTAAACAACGCATTTTTATCGCTGGCCATCGTGG |
| from E. coli (WcaG_EC) | GATGGTCGGTTCCGCCATCACGCGGCAGCTCGAACAGC |
| (corresponding to NCBI- | GCGGTGATGTGGAACTGGTATTACGCACCCGCGACGA |
| ProteinID ADT75652, and | GCTGAACCTGCTGGACAGCCGCGCGGTGCATGATTTCT |
| UniProt E0J126) | TTGCCAGCGAACGCATTGACCAGGTCTATCTGGCGGCG |
| | GCGAAAGTGGGCGGCATTGTTGCCAACAACACCTATCC |
| | GGCGGATTTCATCTACCAGAACATGATGATTGAGAGCA |
| | ACATCATTCACGCCGCGCATCAGAACGACGTGAACAA |
| | ACTGCTGTTTCTTGGATCGTCCTGTATCTACCCGAAACT |
| | GGCAAAACAGCCGATGGCAGAAAGCGAGTTATTGCAG |
| | GGCACGCTGGAGCCGACCAACGAGCCTTACGCCATTGC |
| | CAAAATCGCCGGGATCAAACTGTGCGAATCATACAAC |
| | CGCCAGTACGGACGCGATTACCGCTCAGTCATGCCGAC |
| | CAACCTGTATGGCCCGCATGACAACTTCCACCCGAGTA |
| | ATTCGCATGTGATCCCTGCATTGCTGCGTCGCTTCCACG |
| | AGGCGACGGCACAGAATGCACCGGACGTGGTGGTATG |
| | GGGCAGCGGTACACCGATGCGTGAATTCCTGCACGTCG |
| | ATGATATGGCGGCGGCGAGCATTCATGTCATGGAGCTG |
| | GCGCACGAAGTCTGGCTGGAGAACACCCAGCCGATGC |
| | TGTCGCACATTAACGTCGGCACGGGCGTTGACTGCACT |
| | ATCCGCGAGCTGGCGCAAACCATCGCCAAAGTGGTGG |
| | GTTACAAAGGTCGGGTGGTTTTTGATGCCAGCAAACCG |
| | GATGGTACGCCGCGCAAACTGCTTGATGTGACGCGCCT |
| | GCATCAGCTTGGCTGGTATCACGAAATCTCACTGGAAG |
| | CGGGGCTTGCCAGCACTTACCAGTGGTTCCTTGAGAAT |
| | CAAGACCGCTTTCGGGGGTAA |
| SEQ ID NO 5: Gmd from | ATGATTGACAGAATGGATAAAAACGCAAAAATTTATG |
| Bacteroides fragilis | TAGCCGGACACCACGGACTGGTGGGTTCGGCTATATGG |
| (Gmd_BF) | AAAAATCTGCAGGAAAAGGGGTATACGAATCTGGTGG |
| (corresponding to NCBI- | GACGCACACATAAGGAACTGGACTTATTGGACGGTGC |
| ProteinID CAH07586, and | GACCGTAAAGCAGTTTTTTGATGAGGAAATGCCGGAGT |
| UniProt Q5LE66) | ACGTGTTTTTGGCTGCCGCTTTTGTCGGAGGAATCATG |
| | GCCAATAGTATCTACCGTGCGGACTTTATCTATAAGAA |
| | CTTACAGATACAGCAGAACGTGATCGGAGAAAGTTTCC |
| | GGCATCAGGTGAAAAAACTGCTCTTTCTGGGCAGTACC |
| | TGCATCTATCCGAGGGATGCCGAACAGCCGATGAAAG |
| | AGGACGTCTTGCTCACTTCTCCACTGGAATATACCAAT |
| | GAACCTTATGCCATAGCCAAAATAGCCGGACTGAAAA |
| | TGTGCGAAAGCTTCAACCTGCAATATGGAACGAACTAC |
| | ATCGCCGTGATGCCGACCAACCTGTATGGGCCGAACGA |
| | TAACTTTGACTTGGAACGCAGTCATGTGCTGCCTGCCA |
| | TGATCCGTAAAGTTCACTTGGCACACTGCCTGAAAAAA |
| | GGAGATTGGGAGGCCGTGCGTAAAGATATGAACCTGC |
| | GTCCGGTAGAAGGCATCAGTGGTGCCAACTCCAACGA |
| | AGAGATCCTCCGGATTCTCCGGAAATACGGCATTACTG |
| | AAACGGAGGTGACACTTTGGGGAACAGGAACGCCTCT |
| | AAGGGAATTTCTTTGGAGTGAAGAAATGGCAGATGCC |
| | AGTGTCTTCGTAATGGAACATGTGGACTTCAAGGATAC |
| | CTACAAAACCGGTGCAAAAGACATCCGCAACTGCCAC |
| | ATCAATATAGGTACCGGCAAAGAAATTACAATCCGCG |
| | AACTGGCCGGACTGATTGTAAATACAGTCGGCTATCAG |
| | GGTGAACTGACTTTTGACAGCAGTAAACCGGACGGAA |
| | CCATGCGAAAACTTACCGATCCGTCGAAATTGCACAAC |
| | CTCGGATGGCATCATAAGATCGATATTGAAGAGGGGG |
| | TACAGAAAATGTACGAGTGGTATCTGGGATAA |
| SEQ ID NO 6: WcaG | ATGATTGACAGAATGGATAAAAACGCAAAAATTTATG |
| from Bacteroides fragilis | TAGCCGGACACCACGGACTGGTGGGTTCGGCTATATGG |
| (WcaG_BF) | AAAAATCTGCAGGAAAAGGGGTATACGAATCTGGTGG |
| (corresponding to NCBI- | GACGCACACATAAGGAACTGGACTTATTGGACGGTGC |
| ProteinID CAH07585, and | GACCGTAAAGCAGTTTTTTGATGAGGAAATGCCGGAGT |
| UniProt Q5LE67) | ACGTGTTTTTGGCTGCCGCTTTTGTCGGAGGAATCATG |
| | GCCAATAGTATCTACCGTGCGGACTTTATCTATAAGAA |
| | CTTACAGATACAGCAGAACGTGATCGGAGAAAGTTTCC |
| | GGCATCAGGTGAAAAAACTGCTCTTTCTGGGCAGTACC |
| | TGCATCTATCCGAGGGATGCCGAACAGCCGATGAAAG |
| | AGGACGTCTTGCTCACTTCTCCACTGGAATATACCAAT |
| | GAACCTTATGCCATAGCCAAAATAGCCGGACTGAAAA |
| | TGTGCGAAAGCTTCAACCTGCAATATGGAACGAACTAC |
| | ATCGCCGTGATGCCGACCAACCTGTATGGGCCGAACGA |
| | TAACTTTGACTTGGAACGCAGTCATGTGCTGCCTGCCA |
| | TGATCCGTAAAGTTCACTTGGCACACTGCCTGAAAAAA |
| | GGAGATTGGGAGGCCGTGCGTAAAGATATGAACCTGC |
| | GTCCGGTAGAAGGCATCAGTGGTGCCAACTCCAACGA |
| | AGAGATCCTCCGGATTCTCCGGAAATACGGCATTACTG |
| | AAACGGAGGTGACACTTTGGGGAACAGGAACGCCTCT |
| | AAGGGAATTTCTTTGGAGTGAAGAAATGGCAGATGCC |
| | AGTGTCTTCGTAATGGAACATGTGGACTTCAAGGATAC |
| | CTACAAAACCGGTGCAAAAGACATCCGCAACTGCCAC |
| | ATCAATATAGGTACCGGCAAAGAAATTACAATCCGCG |
| | AACTGGCCGGACTGATTGTAAATACAGTCGGCTATCAG |
| | GGTGAACTGACTTTTGACAGCAGTAAACCGGACGGAA |
| | CCATGCGAAAACTTACCGATCCGTCGAAATTGCACAAC |
| | CTCGGATGGCATCATAAGATCGATATTGAAGAGGGGG |
| | TACAGAAAATGTACGAGTGGTATCTGGGATAA |
| SEQ ID NO 7: FucT from | ATGGCTTTTAAGGTGGTGCAAATTTGCGGGGGGCTTGG |
| H. pylori (HpFucT) | GAATCAAATGTTTCAATACGCTTTCGCTAAAAGTTTGC |
| (corresponding to NCBI- | AAAAACACTCTAATACGCCTGTGCTGTTAGATATTACT |
| ProteinID AAD29863.1, | TCTTTTGATTGGAGTAATAGAAAAATGCAATTAGAACT |
| and UniProt Q9X435) | TTTCCCTATTGATTTGCCCTATGCGAGTGAAAAAGAAA |
| | TCGCTATAGCTAAAATGCAACACCTCCCCAAGCTAGTA |
| | AGAAATGTGCTCAAATGCATGGGGTTTGATAGGGTGA |
| | GTCAAGAAATCGTTTTTGAATACGAGCCTAAATTGTTA |
| | AAGACAAGCCGCTTGACTTATTTTTATGGCTATTTTCAA |
| | GATCCACGATATTTTGATGCTATATCCCCTTTAATCAAG |
| | CAAACCTTCACCCTACCCCCCCCCCCCGAAAATGGAAA |
| | TAATAAAAAAAAAGAGGAAGAATACCACCGCAAGCTT |
| | GCTTTGATTTTAGCCGCTAAAAACAGCGTGTTTGTGCA |
| | TATAAGAAGAGGGGATTATGTGGGGATTGGCTGTCAG |
| | CTTGGTATTGATTATCAAAAAAAGGCGCTTGAGTATAT |
| | GGCAAAGCGCGTGCCAAACATGGAGCTTTTTGTGTTTT |
| | GCGAAGACTTAGAATTCACGCAAAATCTTGATCTTGGC |
| | TACCCTTTTATGGACATGACCACTAGGGATAAAGAAGA |
| | AGAAGCGTATTGGGATATGCTGCTCATGCAATCTTGCA |
| | AGCATGGCATTATCGCTAACAGCACTTATAGCTGGTGG |
| | GCGGCCTATTTGATAAACAATCCAGAAAAAATCATTAT |
| | TGGCCCCAAACACTGGCTTTTTGGGCATGAGAATATCC |
| | TTTGTAAGGAATGGGTGAAAATAGAATCCCATTTTGAG |
| | GTGAAATCCCAAAAGTATAACGCTTAA |
| SEQ ID NO 8: Codon | ATGGCACAGGTCgTGGACTTCAAGATCGTCCAGGTCCA |
| optimized FucT from H. | CGGCGGCCTCGGCAACCAGATGTTCCAGTACGCATTCG |
| mustelae (codon | CAAAGAGCCTCCAGACCCACCTCAACATCCCGGTCCTC |
| optimized HmFucT) | CTCGACACCACCTGGTTCGACTACGGCAACCGTGAGCT |
| (corresponding to NCBI- | CGGCCTCCACCTCTTCCCGATCGACCTCCAGTGCGCAA |
| ProteinID CBG40460.1, | GCGCACAGCAGATCGCAGCAGCACACATGCAGAACCT |
| and UniProt D3UIY5) | CCCGCGTCTCGTCCGTGGCGCACTCCGTCGTATGGGCC |
| | TCGGCCGTGTCAGCAAGGAGATCGTCTTCGAGTACATG |
| | CCGGAGCTCTTCGAGCCGAGCCGTATCGCATACTTCCA |
| | CGGCTACTTCCAGGACCCGCGTTACTICGAGGACATCA |
| | GCCCGCTCATCAAGCAGACCTTCACCCTCCCGCACCOG |
| | ACCGAGCACGCAGAGCAGTACAGCCGTAAGCTCAGCC |
| | AGATCCTCGCAGCAAAGAACAGCGTCTTCGTCCACATC |
| | CGTCGTGGCGACTACATGCGTCTCGGCTGGCAGCTCGA |
| | CATCAGCTACCAGCTCCGTGCAATCGCATACATGGCAA |
| | AGCGTGTCCAGAACCTCGAGCTCTTCCTCTTCTGCGAG |
| | GACCTCGAGTTCGTCCAGAACCTCGACCTCGGCTACCC |
| | GTTCGTCGACATGACCACCCGTGACGGCGCAGCACACT |
| | GGGACATGATGCTCATGCAGAGCTGCAAGCACGGCAT |
| | CATCACCAACAGCACCTACAGCTGGTGGGCAGCATACC |
| | TCATCAAGAACCCGGAGAAGATCATCATCGGCCCGAG |
| | CCACTGGATCTACGGCAACGAGAACATCCTCTGCAAGG |
| | ACTGGGTCAAGATCGAGAGCCAGTTCGAGACCAAGAG |
| | CTAA |
| SEQ ID NO 9: LacY from | ATGTACTATTTAAAAAACACAAACTTTTGGATGTTCGG |
| E. coli (LacY_EC) | TTTATTCTTTTTCTTTTACTTTTTTATCATGGGAGCCTAC |
| (corresponding to NCBI- | TTCCCGTTTTTCCCGATTTGGCTACATGACATCAACCAT |
| ProteinID ADT73957, and | ATCAGCAAAAGTGATACGGGTATTATTTTTGCCGCTAT |
| UniProt E0J0Q8) | TTCTCTGTTCTCGCTATTATTCCAACCGCTGTTTGGTCT |
| | GCTTTCTGACAAACTCGGGCTGCGCAAATACCTGCTGT |
| | GGATTATTACCGGCATGTTAGTGATGTTTGCGCCGTTCT |
| | TTATTTTTATCTTCGGGCCACTGTTACAATACAACATTT |
| | TAGTAGGATCGATTGTTGGTGGTATTTATCTAGGCTTTT |
| | GTTTTAACGCCGGTGCGCCAGCAGTAGAGGCATTTATT |
| | GAGAAAGTCAGCCGTCGCAGTAATTTCGAATTTGGTCG |
| | CGCGCGGATGTTTGGCTGTGTTGGCTGGGCGCTGTGTG |
| | CCTCGATTGTCGGCATCATGTTCACCATCAATAATCAG |
| | TTTGTTTTCTGGCTGGGTTCTGGCTGTGCACTCATCCTC |
| | GCCGTTTTACTCTTTTTCGCCAAAACGGATGCGCCCTCT |
| | TCCGCCACGGTTGCCAATGCGGTAGGTGCCAACCATTC |
| | GGCATTTAGCCTTAAGCTGGCGCTGGAACTGTTCAGAC |
| | AGCCAAAACTGTGGTTTTTGTCACTGTATGTTATTGGC |
| | GTTTCCTGCACCTACGATGTTTTTGACCAACAGTTTGCT |
| | AATTTCTTTACTTCGTTCTTTGCTACCGGTGAACAGGGT |
| | ACGCGGGTATTTGGCTACGTAACGACAATGGGCGAATT |
| | ACTTAACGCCTCGATTATGTTCTTTGCGCCACTGATCAT |
| | TAATCGCATCGGTGGGAAAAACGCCCTGCTGCTGGCTG |
| | GCACTATTATGTCTGTACGTATTATTGGCTCATCGTTCG |
| | CCACCTCAGCGCTGGAAGTGGTTATTCTGAAAACGCTG |
| | CATATGTTTGAAGTACCGTTCCTGCTGGTGGGCTGCTTT |
| | AAATATATTACCAGCCAGTTTGAAGTGCGTTTTTCAGC |
| | GACGATTTATCTGGTCTGTTTCTGCTTCTTTAAGCAACT |
| | GGCGATGATTTTTATGTCTGTACTGGCGGGCAATATGT |
| | ATGAAAGCATCGGTTTCCAGGGCGCTTATCTGGTGCTG |
| | GGTCTGGTGGCGCTGGGCTTCACCTTAATTTCCGTGTTC |
| | ACGCTTAGCGGCCCCGGCCCGCTTTCTCTACTGCGTCG |
| | TCAGGTGAATGAAGTCGCTTAA |
| SEQ ID NO 10: LacY | ATGAAGAAAAAGCTTGTCTCACGCTTGTCGTACGCGGC |
| from Lactobacillus | CGGTGCCTTTGGCAACGACGTCTTCTACGCGACTCTGT |
| delbrueckii (LacY_Ldb) | CAACCTACTTTATCGTCTTCGTCACCACCCACCTCTTTA |
| (corresponding to NCBI- | ATGCCGGTGACCACAAGATGATCTTTATCATCACCAAC |
| ProteinID CAI98004, and | TTGATCACCGCCATCCGGATCGGGGAAGTCCTGCTCGA |
| UniProt P22733) | CCCCTTGATCGGTAACGCCATCGACCGGACCGAAAGCC |
| | GGTGGGGGAAGTTCAAGCCCTGGGTTGTGGGGGGGGG |
| | GATCATCAGCTCATTAGCCCTCTTAGCCCTCTTTACCGA |
| | CTTTGGCGGCATTAACCAAAGCAAACCCGTTGTTTACT |
| | TAGTAATCTTCGGTATTGTTTACTTGATTATGGATATCT |
| | TCTACTCATTTAAAGACACTGGCTTCTGGGCCATGATC |
| | CCGGCCTTGTCCCTGGATTCCCGGGAAAGAGAGAAGA |
| | CCTCCACCTTCGCCAGAGTCGGCTCCACCATCGGGGCC |
| | AACCTGGTCGGGGTAGTCATCACCCCAATCATCCTCTT |
| | CTTCTCGGCTAGCAAGGCCAACCCCAACGGGGATAAG |
| | CAGGGCTGGTTCTTCTTTGCCTTGATCGTGGCCATTGTC |
| | GGCATCTTGACCTCAATTACCGTTGGTCTTGGTACTCAC |
| | GAAGTAAAATCCGCCCTGCGGGAAAGCAATGAAAAGA |
| | CCACTTTGAAGCAGGTCTTTAAGGTCCTGGGGCAAAAC |
| | GACCAGCTCCTCTGGCTGGCCTTTGCCTACTGGTTTTAC |
| | GGCCTGGGTATCAACACCCTGAACGCTCTGCAACTTTA |
| | CTACTTCTCATACATCTTAGGCGATGCCCGCGGCTACA |
| | GCCTGCTTTACACCATCAACACCTTTGTCGGTTTAATCT |
| | CTGCATCCTTCTTCCCATCACTGGCCAAGAAGTTCAAC |
| | AGAAATCGCCTCTTCTACGCCTGCATCGCGGTGATGCT |
| | GTTAGGGATCGGGGTCTTCTCCGTGGCCAGCGGTTCTC |
| | TGGCCCTGTCCCTTGTTGGGGCAGAATTCTTCTTTATTC |
| | CGCAGCCTCTGGCCTTCCTGGTCGTTTTGATGATCATCT |
| | CTGACGCTGTTGAATACGGCCAGCTGAAAACTGGCCAC |
| | AGAGACGAAGCTTTGACCCTGTCTGTCCGGCCATTGGT |
| | CGATAAGCTGGGGGGGGCCTTGTCCAACTGGTTTGTTT |
| | CCTTGATTGCCTTAACTGCCGGCATGACCACTGGGGCG |
| | ACTGCCTCAACAATTACAGCTCATGGCCAGATGGTCTT |
| | CAAGTTAGCTATGTTTGCCTTACCGGCAGTCATGCTCTT |
| | GATCGCTGTTTCTATTTTCGCCAAAAAGGTCTTCTTGAC |
| | TGAAGAAAAGCACGCGGAAATCGTCGACCAGCTGGAA |
| | ACTCAATTCGGCCAAAGCCATGCCCAAAAGCCGGCGC |
| | AAGCTGAAAGCTTCACTTTGGCCAGCCCAGTCTCCGGA |
| | CAATTAATGAACCTGGACATGGTTGACGACCCGGTCTT |
| | TGCCGACAAAAAGTTAGGCGACGGCTTTGCCCTGGTGC |
| | CAGCAGACGGTAAGGTCTACGCGCCATTTGCCGGTACT |
| | GTCCGCCAGCTGGCCAAGACCCGGCACTCGATCGTCCT |
| | GGAAAATGAACATGGGGTCTTGGTCTTGATTCACCTTG |
| | GCCTGGGCACGGCCAAATTAAACGGGACTGGCTTTGTC |
| | AGCTATGTTGAAGAGGGCAGCCAGGTAGAAGCCGGCC |
| | AGCAGATCCTGGAATTCTGGGACCCGGCGATCAAGCA |
| | GGCCAAGCTGGACGACACGGTAATCGTGACCGTCATC |
| | AACAGCGAAACTTTCGCAAATAGCCAGATGCTCTTGCC |
| | GATCGGCCACAGCGTCCAAGCCCTGGATGATGTATTCA |
| | AGTTAGAAGGGAAGAATTAG |
| SEQ ID NO 11: Lacl from | TCACTGCCCGCTTTCCAGTCGGGAAACCTGTCGTGCCA |
| Escherichia Coli | GCTGCATTAATGAATCGGCCAACGCGCGGGGAGAGGC |
| (corresponding to NCBI- | GGTTTGCGTATTGGGCGCCAGGGTGGTTTTTCTTTTCAC |
| ProteinID ADT73959, and | CAGTGAGACGGGCAACAGCTGATTGCCCTTCACCGCCT |
| UniProt A0A0H3EV00) | GGCCCTGAGAGAGTTGCAGCAAGCGGTCCACGCTGGTT |
| | TGCCCCAGCAGGCGAAAATCCTGTTTGATGGTGGTTAA |
| | CGGCGGGATATAACACGAGCTGTCTTCGGTATCGTCGT |
| | ATCCCACTACCGAGATATCCGCACCAACGCGCAGCCCG |
| | GACTCGGTAATGGCGCGCATTGCGCCCAGCGCCATCTG |
| | ATCGTTGGCAACCAGCATCGCAGTGGGAACGATGCCCT |
| | CATTCAGCATTTGCATGGTTTGTTGAAAACCGGACATG |
| | GCACTCCAGTCGCCTTCCCGTTCCGCTATCGGCTGAATT |
| | TGATTGCGAGTGAGATATTTATGCCAGCCCGCCAGACG |
| | CAGACGCGCCGAGACAGAACTTAATGGGCCCGCTAAC |
| | AGCGCGATTTGCTGGTGACCCAATGCGACCAGATGCTC |
| | CACGCCCAGTCGCGTACCGTCTTCATGGGAGAAAATAA |
| | TACTGTTGATGGGAGTCTGGTCAGAGACATCAAGAAAT |
| | AAAGCAGGAACATTAGCGCAGGCTGCTTCCACAGCAA |
| | TGGCATCCTGGTCATCCAGCGGATAGTTAATGATCAGC |
| | CCACTGACGCGTTGCGCGAGAAGGTTGTGCACCGCCGC |
| | TTTACAGGCTTCGACGCCGCTTCGTTCTACCATCGACA |
| | CCACCACGCTGGCACCCAGTTGATCGGCGCGAGATTTA |
| | ATCGCCGCGACAATTTGCGACGGCGCGTGCAGGGCCA |
| | GACTGGAGGTGGCAACGCCAATCAGCAACGACTGTTT |
| | GCCCGCCAGTTGTTGTGCCACGCGGTTGGGAATGTAAT |
| | TCAGCTCCGCCATCGCCGCTTCCACTTTTTCCCGCGTTT |
| | TCGCAGAAACGTGGCTGGCCTGGTTCACCACGCGGGA |
| | AACGGTCTGATAAGAGACACCGGCATACTCTGCGACAT |
| | CGTATAGCGTTACTGGTTTCACATTCACCAC |

The invention therefore encompasses a genetically modified Corynebacterium as described herein comprising an exogenous nucleic acid sequence selected from the group consisting of:
a) a nucleic acid sequence comprising a nucleotide sequence that encodes an enzyme/protein encoded by a nucleotide sequence according to SEQ ID NO 1, 2, 3, 4, 5, 6, 7, 8, 9, 10 or 11;
b) a nucleic acid sequence which is complementary to a nucleotide sequence in accordance with a);
c) a nucleic acid sequence comprising a nucleotide sequence having sufficient sequence identity to be functionally analogous/equivalent to a nucleotide sequence according to a) or b), comprising preferably a sequence identity to a nucleotide sequence according to a) or b) of at least 70%, 80%, preferably 90%, more preferably 95%;
d) a nucleic acid sequence which, because of the genetic code, is degenerated into a nucleotide sequence according to a) through c); and/or
e) a nucleic acid sequence according to a nucleotide sequence of a) through d) which is modified by deletions, additions, substitutions, translocations, inversions and/or insertions and functionally analogous/equivalent to a nucleotide sequence according to a) through d).

Functionally analogous sequences refer to the ability to encode a functional gene product. Functionally analogous sequences refer to the ability to encode a functional gene product and to enable the same or similar functional effect as the gene products of the disclosed sequence.

Function of the proteins/enzyme can be determined by suitable tests for the activity of the respective enzyme/protein, which are routine tests for a skilled person. Appropriate assays for determining enzymatic activity are known in the art.

As used herein, “nucleic acid” shall mean any nucleic acid molecule, including, without limitation, DNA, RNA and hybrids or modified variants thereof. An “exogenous nucleic acid” or “exogenous genetic element” relates to any nucleic acid or nucleic acid sequence introduced into the bacterial cell, which is not a component of the cells “original” or “natural” genome. Exogenous nucleic acids may be integrated or non-integrated, or relate to stably transfected/transformed nucleic acids, and can be an integrative or episomal vector/plasmid containing the respective gene of interest. Accordingly, the terms “exogenous” is used interchangeably with the term “heterologous”, which is known to refers to genetic material that is not part of the host organism.

In preferred embodiments, an exogenous nucleic acid sequence is chromosomally integrated. The term “chromosomal integration” relates to integration of the target genes into the host chromosome, which is a preferable strategy to overcome the drawbacks of plasmid-based overexpression. In bacteria such as for example Escherichia coli, homologous recombination, site-specific recombination and transposon-mediated gene transposition are often used to achieve chromosomal integration.

As used herein, the term expression cassette relates to a distinct component of a nucleic acid sequence, which can for example be comprised by a vector/plasmid DNA or which can be integrated into a host genome. An expression cassette comprises a gene/a coding sequence and regulatory sequence to enable expression of the gene/coding sequence in the genetically modified cell/bacterium. The expression cassette directs the cell's machinery to make RNA and protein(s). An expression cassette can comprise of one or more genes and the sequences controlling their expression. An expression cassette usually comprises three components: a promoter sequence, an open reading frame, and a 3′ untranslated region that. Different expression cassettes can be transfected into different organisms including bacteria, yeast, plants, and mammalian cells as long as the correct regulatory sequences are used.

In embodiments, two or more of the transgenes can be comprised/encoded by a continuous exogenous nucleic acid sequence comprising coding sequences of the two or more transgenes, for example within a single operon. An operon is a functioning unit of DNA containing a cluster of genes, e.g. at least two genes, under the control of a single promoter. The genes are transcribed together into an mRNA strand and either translated together in the cytoplasm or undergo splicing to create monocistronic mRNAs that are translated separately, i.e. several strands of mRNA that each encode a single or multiple gene products. The result of this is that the genes contained in the operon are either expressed together or not at all. Several genes must be co-transcribed to define an operon. Originally, operons were thought to exist solely in prokaryotes (which includes organelles like plastids that are derived from bacteria), but since the discovery of the first operons in eukaryotes in the early 1990s, more evidence has arisen to suggest they are more common than previously assumed. In general, expression of prokaryotic operons leads to the generation of polycistronic mRNAs, while eukaryotic operons lead to monocistronic mRNAs. Operons are also found in viruses such as bacteriophages. For example, T7 phages have two operons. The first operon codes for various products, including a special T7 RNA polymerase which can bind to and transcribe the second operon. The second operon includes a lysis gene meant to cause the host cell to burst.

An operon is made up of several structural genes arranged under a common promoter and regulated by a common operator. It is defined as a set of adjacent structural genes, plus the adjacent regulatory signals that affect transcription of the structural genes. The regulators of a given operon, including repressors, corepressors, and activators, are not necessarily coded for by that operon. The location and condition of the regulators, promoter, operator and structural DNA sequences can determine the effects of common mutations. An operon contains one or more structural genes which are generally transcribed into one polycistronic mRNA (a single mRNA molecule that codes for more than one protein). However, the definition of an operon does not require the mRNA to be polycistronic, though in practice, it usually is. Upstream of the structural genes lies a promoter sequence which provides a site for RNA polymerase to bind and initiate transcription. Close to the promoter lies a section of DNA called an operator.

An operon is made up of 3 basic DNA components: (i.) Promoter, which is a nucleotide sequence that enables a gene to be transcribed. The promoter is recognized by RNA polymerase, which then initiates transcription. In RNA synthesis, promoters indicate which genes should be used for messenger RNA creation—and, by extension, control which proteins the cell produces. (ii.) Operator—a segment of DNA to which a repressor bind. It is classically defined in the lac operon as a segment between the promoter and the genes of the operon. The main operator (O1) in the lac operon is located slightly downstream of the promoter; two additional operators, O1 and O3 are located at −82 and +412, respectively. In the case of a repressor, the repressor protein physically obstructs the RNA polymerase from transcribing the genes. (iii.) Structural genes—the genes that are co-regulated by the operon.

Not always included within the operon, but important in its function is a regulatory gene, a constantly expressed gene which codes for repressor proteins. The regulatory gene does not need to be in, adjacent to, or even near the operon to control it. An inducer (small molecule) can displace a repressor (protein) from the operator site (DNA), resulting in an uninhibited operon. Alternatively, a corepressor can bind to the repressor to allow its binding to the operator site. A good example of this type of regulation is seen for the trp operon.

Suitable promoters for the transgenes of the invention are disclosed herein and include constitutive promotors such as Tuf, GlyA, GroeL, GroES, DnaK, GapA, SOD, PGK and inducible promotors such as Tac, Lac and Tre promoters or synthetic promotors.

For control of expression of the respective enzymes to be expressed in corynebacteria of the invention, constitutive and inducible promoters can be used. As used herein “inducible expression” or “conditional expression” relates to a state, multiple states or system of gene expression, wherein the gene of interest, is preferably not expressed, or in some embodiments expressed at negligible or relatively low levels, unless there is the presence of one or more molecules (an inducer) or other set of conditions in the cell, preferably the Corynebacterium, that allows for gene expression. Inducible promoters may relate to either naturally occurring promoters that are expressed at a relatively higher level under certain biological conditions, or to other promoters comprising any given inducible element. Inducible promoters may refer to those induced by external factors, for example by administration of a small drug molecule or other externally applied signal. An example of such a small molecule used for inducible expression is isopropyl β-d-1-thiogalactopyranoside (IPTG), which is a molecular biology reagent. This compound is a molecular mimic of allolactose, a lactose metabolite that triggers transcription of the lac operon, and it is therefore used to induce protein expression where the gene is under the control of the lac operator. Examples Positively regulated bacterial expression systems have been reviewed by Brautaset et al (Microbial Biotechnology (2009) 2 (1), 15-30).

A transcription terminator is a section of nucleic acid sequence that marks the end of a gene or operon in a DNA sequence during transcription. This sequence mediates transcriptional termination by providing signals in the newly synthesized transcript RNA that trigger processes which release the transcript RNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Suitable examples of terminator sequences that can be used in the context of the invention include any rho-independent terminators such as T7 terminator and a rrnB terminator.

In embodiments, the genetically modified Corynebacterium of the invention is defective for functional expression of one or more glycosyltransferases involved in corynebacterial cell wall biosynthesis.

Almost all Corynebacterium species including Corynebacterium glutamicum are characterized by a complex cell wall architecture: the plasma membrane of these bacteria is covered by a peptidoglycan layer, which itself is covalently linked to arabinogalactan, an additional heteropolysaccharide meshwork. Bound to this, an outer layer of mycolic acids is found which is functionally equivalent to the outer membrane of Gram-negative bacteria. As top layer, outer surface material composed of free polysaccharides, glycolipids, and proteins (including S-layer proteins, pili, and other surface proteins) is found. The general structure and composition of the corynebacterial cell envelope were earlier reviewed by M. Daffé (Handbook of Corynebacterium glutamicum, L. Eggeling and M. Bott, Eds., pp. 121-148, Taylor & Francis, Boca Raton, Fla, USA, 2005) and L. Eggeling et al. (Corynebacteria, A. Burkovski, Ed., pp. 267-294, Caister Academic Press, Norfolk, UK, 2008).

Glycosyltransferases are (GTFs, Gtfs) are enzymes that establish natural glycosidic linkages. They catalyze the transfer of saccharide moieties from an activated nucleotide sugar (also known as the “glycosyl donor”) to a nucleophilic glycosyl acceptor molecule, the nucleophile of which can be oxygen-carbon-, nitrogen-, or sulfur-based. The result of glycosyl transfer can be a carbohydrate, glycoside, oligosaccharide, or a polysaccharide. Some glycosyltransferases catalyse transfer to inorganic phosphate or water. Glycosyl transfer can also occur to protein residues, usually to tyrosine, serine, or threonine to give O-linked glycoproteins, or to asparagine to give N-linked glycoproteins. Mannosyl groups may be transferred to tryptophan to generate C-mannosyl tryptophan, which is relatively abundant in eukaryotes. Transferases may also use lipids as an acceptor, forming glycolipids, and even use lipid-linked sugar phosphate donors, such as dolichol phosphates.

Glycosyltransferases (GTs) are known to be critically involved in the bacterial cell wall biosynthesis, in particular in Corynebacterium. In particular, GTs are involved in the construction of the peptidoglycan layer of the Corynebacterium cell wall. Peptidoglycan is an essential polymer that forms a protective shell around bacterial cell membranes. The peptidoglycan layer surrounds the cytoplasmic membrane of Corynebacteria and functions as an exoskeleton, maintaining cell shape and stabilizing the membrane against fluctuations in osmotic pressure. Peptidoglycan is synthesized in an intracellular phase in which UDP-N-acetylglucosamine is converted to a diphospholipid-linked disaccharide-pentapeptide known as Lipid II, and in an extracellular phase in which the disaccharide (NAG-NAM) subunits of translocated Lipid II are coupled by peptidoglycan glycosyltransferases (PGTs; also known as transglycosylases) to form linear carbohydrate chains, which are cross-linked through the attached peptide moieties by transpeptidases. PGTs are defined by the presence of five conserved sequence motifs and exist in two forms: (i) as N-terminal glycosyltransferase domains in bifunctional proteins that also contain a C-terminal transpeptidase domain [called class A penicillin-binding proteins (PBPs)], and (ii) as monofunctional proteins (MGTs) that do not contain transpeptidase domains. Different bacteria typically contain different numbers and types of PGTs, and it is thought that the different PGTs play different roles during the bacterial cell cycle, with some involved primarily in cell elongation and others recruited to the septal region during cell division. Regardless of their cellular roles, all PGTs catalyze glycosyltransfer from a polyprenyl-diphosphate moiety on the anomeric center of an N-acetyl muramic acid (NAM) unit to the C4 hydroxyl of an N-acetylglucosamine (NAG) moiety. The mechanism of glycosyltransfer is not well understood, and it is taking considerable effort to identify soluble, well behaved PGT domains to use as model systems for detailed mechanistic and structural analysis.

In the context of the present invention, it was surprisingly found that Corynebacteria and in particular Corynebacterium glutamicum with deficiency for one of more GTs leads to a markedly reduced foam production during the culturing process and also increases the yield of biomolecules produced by the bacteria, such as fucosyllactose.

In embodiments, the Corynebacterium glutamicum is defective for functional expression of one or more glycosyltransferases, preferably selected from the group comprising GTs comprising cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

In embodiments, the Corynebacterium glutamicum is defective for functional expression of one or more glycosyltransferases, preferably selected from the group comprising GTs comprising cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_1180 (GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); egp_2385 (GT87); and cgp_3164.

In embodiments, the Corynebacterium glutamicum is defective for functional expression of one or more glycosyltransferases, preferably selected from the group comprising GTs comprising cgp_3313 (MrcB; GT51), cgp_0336 (PonA; GT51); egp_3166 (GT4); cgp_2400 (GT4); cgp_1876 (GT4); cgp_1268 (GlgA; GT4); cgp_0554 (GT4); cgp_3191 (GlfT; GT2); cgp_1672 (PpmC; GT2); cgp_2393 (GT87,GT87); cgp_2390 (GT87); cgp_2389 (GT87); cgp_2385 (GT87); and cgp_3164.

In embodiments, the present invention relates to a method of producing biomolecules, such as fucosyllactose, the method comprising culturing a genetically modified Corynebacterium of the present invention in a medium supplemented with lactose. In such methods, the bacteria can be cultivated, for example, in shake flask cultivation or Fedbatch cultivation, as is well established in the art. The method of cultivating the bacteria of the invention for the production of biomolecules can be performed according to well established protocols of the state of the art, which are known to the skilled person and which are disclose in the literature, for example in the Handbook of Corynebacterium glutamicum (Edited By Lothar Eggeling, Michael Bott; doi.org/10.1201/9781420039696) or in Corynebacterium Glutamicum: Biology and Biotechnology (edited by Tatsumi, Nami, Inui, Masayuki; Springer; 2013. Edition (14 Aug. 2012)).

In embodiments, the bacteria of the invention are cultured in a fed-batch process. Fed-batch culture is, in the broadest sense, defined as an operational technique in biotechnological processes where one or more nutrients (substrates) are fed (supplied) to the bioreactor during cultivation and in which the product(s) remain in the bioreactor until the end of the run. An alternative description of the method is that of a culture in which a base medium supports initial cell culture and a feed medium is added to prevent nutrient depletion. It is also a type of semi-batch culture. In some cases, all the nutrients are fed into the bioreactor. The advantage of the fed-batch culture is that one can control concentration of fed-substrate in the culture liquid at arbitrarily desired levels (in many cases, at low levels). Generally speaking, fed-batch culture is superior to conventional batch culture when controlling concentrations of a nutrient (or nutrients) affects the yield or productivity of the desired metabolite.

In the context of the invention, fed-batch culture can be performed as described in this paragraph, representing a preferred embodiment. The Fed-batch culture can be performed in a 1-L fermenter (Multifors, Infors) with 0.4 L working volume. The seed cultures may be prepared in CGXII medium in shake flasks as described herein; the fermenter can be inoculated to an OD600 of 1 in CGXII medium with 4% w/v glucose. The agitation and aeration are preferably set at 800 rpm and 1 vvm, respectively. The pH is maintained at 7.0 and temperature at 30° C. throughout the fermentation. Dissolved oxygen (DO) is measured by an electrode, 100% is set by oxygen-saturated distilled water. 1 mM IPTG is added at t=16 h and 4 g/L lactose is added at t=25 h. Feeding medium contained 400 g/L glucose and 100 g/L (NH4)2SO4, and the feed frequency is variably adjusted automatically depending on the DO signal. The feeding starts when DO exceeds 30% and stops when DO felt again under the set-point.

In preferred embodiments of the methods of the invention, the Corynebacteria are cultured in the presence of lactose and glucose in order to provide the substrates for the fucosyllactose biosynthesis pathway comprised by bacteria of the invention.

In embodiments of the method of the invention, the bacteria of the invention are cultured in a shake flask cultivation. In such embodiments, all shake flasks cultivations can be performed at 30° C. at agitation speed of 200 rpm. Cells cultured on BHIS agar plates for 2 days are sequentially transferred into 10 ml of the LBG medium (yeast extract: 5 g/L, peptone: 10 g/L, NaCl: 10 g/L, glucose: 1 g/L) grown overnight, and then to a 500 mL flask containing 150 mL of CGXII medium (20 g/L (NH4)2SO4, 5 g/L urea, 1 g/L KH2PO4, 1 g/L K2HPO4, 0.25 g/L MgSO4·7H2O, 10 mg/L CaCl2, 10 mg/L FeSO4·7H2O, 10 mg/L MnSO4·H2O, 1 mg/L ZnSO4·7H2O, 0.2 mg/L CuSO4·5H2O, 0.02 mg/L NiCl2·6H2O, 0.2 mg/L biotin, 30 mg/L 3,4-dihydroxybenzoic acid, and 21 g/L 3-morpholinopropanesulfonic acid (MOPS); pH 7.0) with 4% w/v glucose. 1 mM isopropyl-β-D-thiogalactoside (IPTG) was added after 4 h, additionally, 1 g/L Lactose was supplemented to the strains after 8 h of inoculation.

### EXAMPLES

The invention is further described by the following examples. These are not intended to limit the scope of the invention but represent preferred embodiments of aspects of the invention provided for greater illustration of the invention described herein.

### Methods Employed in the Examples

Genetically engineered C. glutamicum strains generated and used in the examples.

The following strains of C. glutamicum were used in the present examples (see Table 2). The strain MB001 was purchased from DSMZ, while the other strains representing examples of the invention have been generated as explained below.

TABLE 2
Strains used in the examples.

| Strain | Relevant characteristics | |
|---|---|---|
| MB001 | ATCC 13032 with Prophages - | Baumgart, |
| | CGP1, CGP2, and CGP3 deleted | M. et al. |
| | | (2013). |
| MB02 | MB001 - Δcgp_2725 ::P Tac-GMD_WcaG- P | This work |
| | Trc-HpFucT, Δcgp_1213 ::P Tac- | |
| | ManB_ManC, Δcgp_2854 ::P GlyA-LdbLacY, | |
| | Δcgp_3151 ::P Laciq-LacI; with integration of | |
| | single copy genes under regulated promotors - | |
| | ManC_ManB GMD_WcaG operons from | |
| | Escherichia coli under IPTG inducible Tac | |
| | promotor, fucosyltransferase HpFucT gene | |
| | from Helicobacter pylori under IPTG inducible | |
| | Tre promotor, Lactose permease gene | |
| | LdbLacY from Lactobacillus delbrueckii under | |
| | growth phase regulated corynebacterial GlyA | |
| | promotor and Lactose repressor gene LacI | |
| | under LacIq promotor) | |
| MB03 | MB02 Δcgp_1178::P Tuf-CO-HmFucT; codon | This work |
| | optimized FucT from Helicobacter mustulae | |
| | driven under strong constitutive promotor Tuf | |
| | integrated in MB02. | |
| MB04 | MB03 Δcgp_1782 ::P Tuf-LacO-WcaG-GMD; | This work |
| | genes WcaG and GMD from Bacteriodes | |
| | Fraglis under constitutive promotor under Lac | |
| | operator integrated in MB03. | |
| MB07 | MB04 Δcgp_2600::P LacUV5-LacY; LacY | This work |
| | gene from Escherichia coli under unregulated | |
| | promotor integrated in MB04. | |
| MB13 | MB07 Δcgp_0336; deletion of gene in MB07 | This study |
| | involved in the synthesis of peptidoglycan | |
| | layer of cell wall. | |
| MB14 | MB07 Δcgp_3191, deletion of gene in MB07 | |
| | involved in the synthesis of plasma membrane | |
| | of cell wall. | |
| MB15 | MB07 Δcgp_3164; deletion of gene in MB07 | This study |
| | involved in the synthesis of plasma membrane | |
| | of cell wall. | |
| MB17 | MB07 Δcgp_0554; deletion of gene in MB07 | This study |
| | involved in the synthesis of outer membrane | |
| | glycolipids of cell wall. | |
| MB20 | MB07 Δcgp_2385; deletion of gene in MB07 | This study |
| | involved in the synthesis of outer membrane | |
| | glycolipids of cell wall. | |
| MB11 | MB07 Δcgp_1672; deletion of gene in MB07 | This study |
| | involved in the synthesis of outer membrane | |
| | glycolipids of cell wall. | |
| MB21 | MB07 Δcgp_2400; deletion of gene in MB07 | This study |
| | involved in the synthesis of outer membrane | |
| | glycolipids of cell wall. | |
| MB22 | MB07 Δcgp_1876; deletion of gene in MB07 | This study |
| | involved in the synthesis of outer membrane | |
| | glycolipids of cell wall. | |
| MB23 | MB07 Δcgp_2393; deletion of gene in MB07 | This study |
| | involved in the synthesis of outer membrane | |
| | glycolipids of cell wall. | |
| MB24 | MB07 Δcgp_2390; deletion of gene in MB07 | This study |
| | involved in the synthesis of outer membrane | |
| | glycolipids of cell wall. | |
| MB26 | MB07 Δcgp_3313; deletion of gene in MB07 | This study |
| | involved in the synthesis of outer membrane | |
| | glycolipids of cell wall. | |
| MB27 | MB07 Δcgp_2389; deletion of gene in MB07 | This study |
| | involved in the synthesis of outer membrane | |
| | glycolipids of cell wall. | |
| MB143 | MB07 Δcgp_1672 Δcgp_0336; deletion of | This study |
| | gene in MB07 involved in the synthesis of outer | |
| | membrane glycolipids of cell wall. | |
| MB195 | MB07 Δcgp_3164 Δcgp_0336; deletion of | This study |
| | gene in MB07 involved in the synthesis of outer | |
| | membrane glycolipids of cell wall. | |

The transgenes of Table 3 have been used for constructing the strains of Table 2.

TABLE 3
Genes used in transgenic strains of the invention.

| | NCBI | |
|---|---|---|
| Genes | ProteinID | UniProt |
| ManB | ADT75633 | E0J145 |
| ManC | ADT75634 | E8PZ98 |
| GMD_EC | ADT75653 | E0J125 |
| WcaG_EC | ADT75652 | E0J126 |
| GMD, BF | CAH07586 | Q5LE66 |
| WcaG_BF | CAH07585 | Q5LE67 |
| Lactose Permease_EC | ADT73957 | E0J0Q8 |
| Lactose | CAI98004 | P22733 |
| Permease_LDB | | |
| HPfucT | AAD29863.1 | Q9X435 |
| HMfucT | CBG40460.1 | D3UIY5 |

The genetic modification of the strains was carried out using the following protocol:

Chromosomal modifications are carried out by homologous recombination. Expression cassettes to be integrated or genes to be deleted were cloned together with about 1000 bp up- and downstream of chromosomal regions into vector suicide vector pK19mobsacB bearing kanamycin gene for antibiotic selection and a lethal gene levansucrase from Bacillus subtilis. Following transformation, clones with integrated vector were selected on BHIS agar plates with 25 μg/ml kanamycin in the first step. Selected clones grown in 3 ml BHIS at 30° C., 200 rpm overnight were plated at different dilutions on LB agar plates with 10% (w/v) sucrose, LB agar plates with 10% (w/v) sucrose and 25 μg/ml Kanamycin and LB agar plates with 25 μg/ml Kanamycin. Clones obtained on sucrose-plates were further checked for expression cassettes integration or deletion by PCR.

### Shake Flask Cultivation.

All shake flasks cultivations were performed at 30° C. under agitation speed of 200 rpm. Cells cultured on BHIS agar plates for 2 days were sequentially transferred into 10 ml of the LBG medium (yeast extract: 5 g/L, peptone: 10 g/L, NaCl: 10 g/L, glucose: 1 g/L) grown overnight, and then to a 500 mL flask containing 150 mL of CGXII medium (20 g/L (NH₄)₂SO₄, 5 g/L urea, 1 g/L KH₂PO₄, 1 g/L K₂HPO₄, 0.25 g/L MgSO₄·7H₂O, 10 mg/L CaCl₂), 10 mg/L FeSO₄·7H₂O, 10 mg/L MnSO₄·H₂O, 1 mg/L ZnSO₄·7H₂O, 0.2 mg/L CuSO₄·5H₂O, 0.02 mg/L NiCl·₆H₂O, 0.2 mg/L biotin, 30 mg/L 3,4-dihydroxybenzoic acid, and 21 g/L 3-morpholinopropanesulfonic acid (MOPS); pH 7.0) with 4% w/v glucose. 1 mM isopropyl-β-D-thiogalactoside (IPTG) was added after 4 h, additionally, 1 g/L Lactose was supplemented to the strains after 8 h of inoculation.

### Fed-Batch Cultivation

Fed-batch culture was performed in a 1-L fermenter (Multifors, Infors) with 0.4 L working volume. The seed cultures were prepared in CGXII medium in shake flasks as described earlier; the fermenter was inoculated to an OD₆₀₀ of 1 in CGXII medium with 4% w/v glucose. The agitation and aeration were set at 800 rpm and 1 vvm, respectively. The pH was maintained at 7.0 and temperature at 30° C. throughout the fermentation. Dissolved oxygen (DO) was measured by an electrode, 100% was set by oxygen-saturated distilled water. 1 mM IPTG was added at t=16 h and 4 g/L lactose was added at t=25 h. Feeding medium contained 400 g/L glucose and 100 g/L (NH₄)₂SO₄, and the feed frequency was variably adjusted automatically depending on the DO signal. The feeding started when DO exceeds 30% and stops when DO felt again under the set-point.

Integration of Lactose Permease in C. glutamicum

Lactose is the penultimate substrate of synthetic pathways involved in the synthesis of simple HMOs and thus lactose uptake is a primary requirement for the production of HMOs in the chassis strain. To assimilate whey, lactose permease (LacY), and beta-galactosidase (LacZ) from E. coli and Lactobacillus delbrueckii subsp. Bulgaricus were expressed in C. glutamicum under the IPTG-inducible tac promoter in shuttle vectors (Barrett et al., 2004; Brabetz et al., 1991). Plasmid based expression of these genes from two both gram positive and gram-negative species resulted in efficient lactose assimilation, but genome integrated cassettes of these operons from IPTG-inducible P LacUV5 resulted in indolent growth on lactose. Accordingly, the inventors reasoned that Lactose permease expression was insufficient for lactose uptake or its assimilation. Therefore, the inventors attempted to express Lactose permease under the control of promotors of different strength to identify strains for efficient lactose uptake.

Lactose permease (LacY) expression cassettes under different promotors were integrated by deleting Cgp_2854 transposase in the genome of MB001 employing integration vector pK19mobsacB-cgp2856_PxxxLacY_cgp2853. Five promotors (Pxxx) of different strengths were selected, two IPTG inducible heterologous promotors (P_{lac} and P_{tac}) and three constitute promotors of C. glutamicum (P_{GlyA}, P_{sod}, P_{Tuf}). The LacY expressing downstream promotors of different strengths were integrated in the genome and the strains were designated as Strain_TAC, Strain_LAC, Strain_SOD, Strain_GLY, and Strain_TUF respectively.

The resulting strains were characterized based on the rate of lactose assimilation from media relative to glucose assimilation. pEKEX2 containing the beta-Galactosidase (LacZ) expressing shuttle vector under IPTG inducible Ptac promotor was cloned into respective strains. The strains were grown in CGXII media with 2% glucose as carbon source with 1 mM Isopropyl β-D-1-thiogalactopyranoside (IPTG). After 24 hours, cultures were diluted to OD600 of 0.6 to 0.8 in CGXII media with 2% Lactose as sole carbon source containing 1 mM IPTG to monitor comparative growth.

Strain_Tac and Strain_GlyA showed comparable growth. Strain_Tuf was not amenable for further electroporation, probably due to over expression of LacY that might have weakened the cellular membrane. While Strain_Lac and Strain_Sod could not support the growth on Lactose as carbon source despite the expression of LacZ from a strong promotor. Strain_GLY was further used to construct the production strain. A cassette encoding Lac repressor (LacI) under the strong promoter PLaciq was integrated into the chromosomal location, deleting cgp_3151. The resulting strain is designated MBP001.

Integration of Lac Operon from E. coli at Different Chromosomal Sites in C. glutamicum

Chromosomal positions affect gene expression in both eukaryotes and prokaryotes. In mouse embryonic stem cells, reporter gene expression varied more than 1000-fold across the genome based on an analysis of 27000 positions. In E. coli, expression of a reporter gene cassette driven by the E. coli lac promoter varied by a factor of 300 depending on chromosomal position and was completely repressed at certain positions. The differences in gene expression occurred primarily at the transcriptional level, which is mediated by pleiotropic effects in chromosome organization. Similar studies did not exist for C. glutamicum. Data were collected using a reporter cassette expressing LacZ and LacY genes integrated into different genomic regions targeting transposase gene deletion.

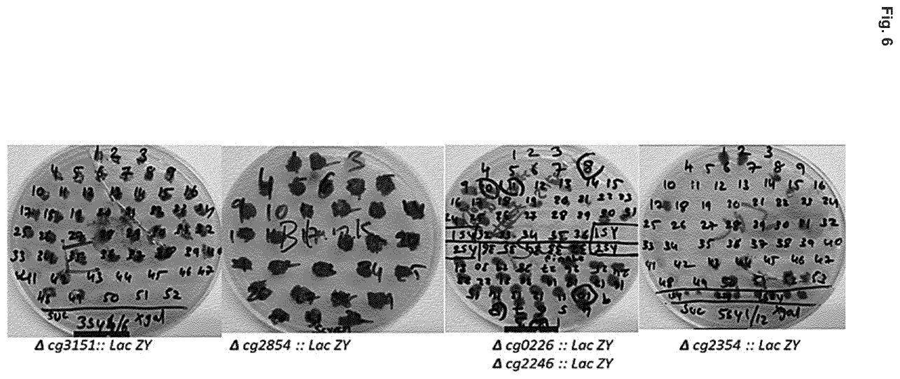

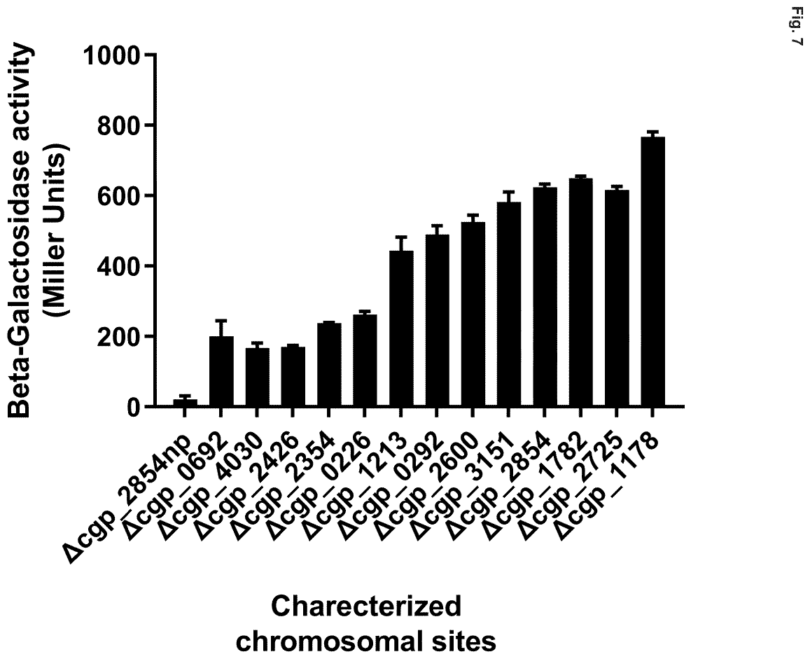

The pK19Sac vector is a modified pK19MobSac vector without the LacZ alpha fragment, Lac promoter, and RP4 mob DNA regions. Therefore, the resulting vector is smaller than pK19MobSac. To construct a deletion vector, ˜1000 bp segments homologous to chromosomal targets immediately upstream and downstream of the integration site were amplified by PCR and incorporated into the EcoRI- and HindIII-digested pK19Sac vector with a SmaI or PmeI restriction site in between using the NEBuilder HiFi DNA Assembly Kit (New England Biolabs). The vectors are designated as pk19Sac-Δcgpxxxx (xxxx is the deletion site number). For example, LacZ and LacY genes with native Lac promoter were amplified from E. coli W genomic DNA and cloned into vectors digested with SmaI. LacZ and LacY without promoter were cloned into regions flanking the cgp_2854 gene. The resulting vectors were transformed into MB001 by electroporation. Recombinants of kanamycin plates grown overnight in BHI media were diluted and plated on LB plates containing 10% sucrose. Positive candidates identified by blue/white screening on X-gal plates were examined by colony PCR to confirm removal of levansucrase (SacB) and kanamycin resistance cassette (FIG. 6). Beta-Galactosidase expression was quantified by a Miller Assay (Miller, 1972) to characterize the ideal chromosomal sites for expression (FIG. 7).

### Measurement of Intracellular and Extracellular 2′FL

2 FL production was monitored by separating cells from the medium at different time points with 1-ml samples by centrifugation at 13,000 g for 10 minutes. The supernatant is an extracellular fraction. The pellets were washed three times in phosphate buffer saline, suspended in 1 ml CGXII medium in a microcentrifuge tube, heated at 100° C. for 30 minutes, cooled on ice, shaken, and centrifuged at 13,000*g for 10 minutes. Standards were used to calibrate and determine the concentrations of the components in the samples.

To compare the extracellular and intracellular concentrations of 2 FL with the cell culture volume, the cell culture volume from which the cells were taken was normalized. Intracellular and extracellular fractions labeled 2 AB were analyzed by HPLC using TSKgel Amide-80 column (5 μm, 80 Å, 250× 4.6 mm i.d., from Tosoh Biosep, Stuttgart, Germany; Bigge J C, et al., Non-selective and efficient fluorescent labeling of glycans with 2-aminobenzamide and anthranilic acid. Anal Biochem. 1995 Sep. 20; 230 (2): 229-38.). For all separations, HPLC grade water with acidic pH was used as solvent A and 100% acetonitrile (ACN) as solvent B with a gradient from 80 to 50% ACN for 35 min. The 2-AB derivatives were excited at 330 nm and emission was measured at 420 nm using fluorescence detector.

Integration of Expression Cassettes in C. glutamicum to Construct MB07

The donor plasmids generated used different promoters and terminators cloned into the pUC19 vector. A short, Rho-independent terminator was designed upstream of the promoter in the donor vectors. PTac promoter and T7 terminator yield p19TacT7, PLacUV5 promoter and T7 terminator yield p19TacT7, PLac promoter and T7 terminator yield p19LacT7, PTre promoter and ribosomal terminator yield p19TrcRRNT, and PTuf and T7 terminator yield p19TufT7.

In p19TacT7, an operon encodes for, GDP-mannose 4,6-dehydrogenase (GMD) and GDP-4-keto-6-deoxy-D-mannose 3,5-epimerase/4-reductase (FCI) from Escherichia coli was cloned under the PTac promoter in p19TacT7. Fucosyltransferase amplified from Helicobacter pylori was cloned under the PTrc promoter in p19TrcRRNT. Cassettes of PTac-GMD_FCI-T7T and Trc-HpFucT-rrnT from the above constructs were amplified and assembled into PmeI-digested pk19Sac-Δcgp_2725 using the NEBuilder HiFi DNA Assembly Kit and transformed into Strain_GLY by electroporation. The resulting strain is MBP002.

An operon encoding phosphomannomutase (ManB), mannose-1-phosphate guanylyltransferase (ManC) from Escherichia coli was cloned into p19TacT7 under PTac.

The gene cassette Ptac-ManB_ManC-T7T from the resulting donor vector was amplified by PCR and cloned into the PmeI-digested pk19Sac-Δcgp_1213 vector, which is shown in MB02. Synthetic codon-optimized fucosyltransferase from Helicobacter mustulae was cloned into PTuf at p19TufT7, and the resulting cassette PTuf-COHmFucT-T7 was cloned into the vector pk19Sac-Δcgp_1178 digested with PmeI. MB02 was transformed with the vector to generate strain MB03.

FCI and GMD of Bacteroides fraglis were transformed under the PTuf transcription start site with LacO in p19TufT7. The PTufLacO-FCI_GMD-17 expression cassette was inserted into the PmeI-digested pk19Sac-Δcgp_1782 clone, transformed into MB03, and strain MB04 was constructed.

To further improve lactose uptake, MB07 was transformed with lactose permease from the E. coli W strain that was expressed under the PLacUV5 promoter in MB04, with cgp_2600 deleted to generate MB07. Further strains were constructed by deleting glycosyltransferases involved in cell wall synthesis (see Table 2).

1. A genetically modified or engineered Corynebacterium for production of fucosyllactose, wherein the Corynebacterium is defective for functional expression of one or more glycosyltransferases involved in corynebacterial cell wall biosynthesis.

2. The genetically modified Corynebacterium according to claim 1, wherein the one or more glycosyltransferases is selected from the group consisting of cgp_3313 (MrcB; GT51), cgp_0336 (PonA; GT51); cgp_3166 (GT4); cgp_2400 (GT4); cgp_1876 (GT4); cgp_1268 (GlgA; GT4); cgp_0554 (GT4); cgp_3191 (GIFT; GT2); cgp_1672 (PpmC; GT2); cgp_1180 (GT2); cgp_0848 (WbbL; GT2); cgp_0730 (GT2); cgp_0396 (GT2); cgp_0394 (GT2); cgp_0246 (GT2); cgp_0163 (GT2); cgp_2393 (GT87,GT87); cgp_2390 (GT87); cgp_2389 (GT87); cgp_2385 (GT87); and cgp_3164.

3. The genetically modified Corynebacterium according to claim 1, wherein the Corynebacterium is Corynebacterium glutamicum.

4. The genetically modified Corynebacterium according to the claim 1, wherein the Corynebacterium has been modified to express a permease for lactose import from exogenous nucleic acid sequences.

5. (canceled)

6. The genetically modified Corynebacterium according to claim 4, wherein the Corynebacterium additionally comprises exogenous nucleic acids for expression of phosphomannomutase (ManB) and GTP-mannose-1-phosphate guanylyltransferase (ManC).

7. The genetically modified Corynebacterium according to claim 1, where the Corynebacterium exports fucosyllactose.

8. The genetically modified Corynebacterium according to claim 4, wherein the permease for lactose import is selected from a group including lactose permease (LacY)), GDP-D-mannose-4,6-dehydratase (GMD), GDP-L-fucose synthase (WcaG), and fucosyltransferase (FucT).

9. The genetically modified Corynebacterium according to claim 6, wherein the exogenous nucleic acids for expression of phosphomannomutase (ManB) and GTP-mannose-1-phosphate guanylyltransferase (ManC) are chromosomally integrated.

10. The genetically modified Corynebacterium according to claim 8, wherein the exogenous nucleic acid sequences for expression of LacY, GMD, WcaG, or FucT are chromosomally integrated.
